(19) United States
(12) Patent Application Publication
(10) Pub. No.: US 2024/0277410 A1
(43) Pub. Date: Aug. 22, 2024

(54) INTRAVASCULAR LITHOTRIPSY DEVICE THAT GENERATES AN ADVANCING ENERGY WAVEFRONT

(71) Applicant: Bolt Medical, Inc., Carlsbad, CA (US)

(72) Inventor: Christopher A. Cook, Laguna Niguel, CA (US)

(21) Appl. No.: 18/653,148

(22) Filed: May 2, 2024

Related U.S. Application Data
(63) Continuation-in-part of application No. 17/146,867, filed on Jan. 12, 2021.
(60) Provisional application No. 62/964,529, filed on Jan. 22, 2020.

(51) Int. Cl. A61B 18/24; A61B 18/00

(52) U.S. Cl. CPC A61B 18/245; A61B 2018/00172; A61B 2018/00184; A61B 2018/0022; A61B 2018/0041; A61B 2018/00702; A61B 2018/00732; A61B 2018/00761

(57) ABSTRACT

A catheter system (100) for treating a treatment site (106) includes a catheter shaft (110), a balloon (104), an energy source (124), a plurality of energy guides (122A), and a system controller (126). The energy source (124) generates pulses of energy. The energy guides (122A) are each configured to selectively receive at least one of the pulses of energy. Each of the energy guides (122A) includes a guide distal end (122D) that is positioned within a balloon interior (146) at a different longitudinal position from one another along a length (142) of the balloon (104). The system controller (126) (i) controls at least one of a pulse frequency, a pulse energy level and a pulse width of each of the pulses of energy, and (ii) controls a firing sequence of the pulses of energy such that the pulses of energy are sequentially directed to each of the energy guides (122A), so that an advancing wavefront (835) is generated within the balloon interior (146) that moves toward the treatment site (106).

### RELATED APPLICATIONS

This application is a continuation-in-part application of and claims priority on U.S. patent application Ser. No. 17/146,867, filed on Jan. 12, 2021. Additionally, U.S. patent application Ser. No. 17/146,867 claims priority on U.S. Provisional Patent Application Ser. No. 62/964,529, filed on Jan. 22, 2020. As far as permitted, the contents of U.S. patent application Ser. No. 17/146,867, and U.S. Provisional Patent Application Ser. No. 62/964,529, are incorporated in their entirety herein by reference.

### BACKGROUND

Vascular lesions within vessels in the body can be associated with an increased risk for major adverse events, such as myocardial infarction, embolism, deep vein thrombosis, stroke, and the like. Severe vascular lesions can be difficult to treat and achieve patency for a physician in a clinical setting.

Vascular lesions may be treated using interventions such as drug therapy, balloon angioplasty, atherectomy, stent placement, vascular graft bypass, to name a few. Such interventions may not always be ideal or may require subsequent treatment to address the lesion.

### SUMMARY

The present invention is directed toward a catheter system for treating a treatment site within or adjacent to a vessel wall of a blood vessel. In various embodiments, the catheter system includes a catheter shaft, a balloon, an energy source, a plurality of energy guides, and a system controller. The balloon is coupled to the catheter shaft. The balloon includes a balloon first end, a balloon second end, and a balloon wall that defines a balloon interior. The balloon is configured to be positioned adjacent to the treatment site. The energy source generates a plurality of pulses of energy. Each of the plurality of energy guides is configured to selectively receive at least one of the plurality of pulses of energy from the energy source. Each of the plurality of energy guides is disposed along the catheter shaft and at least partially within the balloon interior. The plurality of energy guides each include a corresponding guide distal end. Each of the guide distal ends is positioned within the balloon interior at a different longitudinal position from one another along a length of the balloon. The system controller (i) controls at least one of a pulse frequency, a pulse energy level and a pulse width of each of the plurality of pulses of energy from the energy source, and (ii) controls a firing sequence of the plurality of pulses of energy from the energy source such that the plurality of pulses of energy from the energy source are sequentially directed to each of the plurality of energy guides, so that an advancing wavefront is generated within the balloon interior that moves toward the treatment site in a first direction from near the balloon first end toward the balloon second end.

In some embodiments, the system controller controls at least two of the pulse frequency, the pulse energy level and the pulse width of each of the plurality of pulses of energy from the energy source, so that the advancing wavefront is generated within the balloon interior that moves toward the treatment site in the first direction from near the balloon first end toward the balloon second end.

In certain embodiments, the system controller controls each of the pulse frequency, the pulse energy level and the pulse width of each of the plurality of pulses of energy from the energy source, so that the advancing wavefront is generated within the balloon interior that moves toward the treatment site in the first direction from near the balloon first end toward the balloon second end.

In some embodiments, the system controller controls the pulse frequency so that the pulse frequency is increasing during the firing sequence of the plurality of pulses of energy from the energy source.

In other embodiments, the system controller controls the pulse frequency so that the pulse frequency is decreasing during the firing sequence of the plurality of pulses of energy from the energy source.

In certain embodiments, the system controller controls the pulse energy level so that the pulse energy level is increasing during the firing sequence of the plurality of pulses of energy from the energy source.

In other embodiments, the system controller controls the pulse energy level so that the pulse energy level is decreasing during the firing sequence of the plurality of pulses of energy from the energy source.

In some implementations, the system controller controls the energy source so that the pulse frequency is increasing and the pulse energy level of each of the plurality of pulses of energy is increasing during the firing sequence, so that the advancing wavefront is generated within the balloon interior that moves toward the treatment site in the first direction from near the balloon first end toward the balloon second end.

In other implementations, the system controller controls the energy source so that the pulse frequency is increasing and the pulse energy level of each of the plurality of pulses of energy is decreasing during the firing sequence, so that the advancing wavefront is generated within the balloon interior that moves toward the treatment site in the first direction from near the balloon first end toward the balloon second end.

In still other implementations, the system controller controls the energy source so that the pulse frequency is decreasing and the pulse energy level of each of the plurality of pulses of energy is increasing during the firing sequence, so that the advancing wavefront is generated within the balloon interior that moves toward the treatment site in the first direction from near the balloon first end toward the balloon second end.

In yet other implementations, the system controller controls the energy source so that the pulse frequency is decreasing and the pulse energy level of each of the plurality of pulses of energy is decreasing during the firing sequence, so that the advancing wavefront is generated within the balloon interior that moves toward the treatment site in the first direction from near the balloon first end toward the balloon second end.

In many embodiments, the catheter system further includes a multiplexer that receives the plurality of pulses of energy from the energy source; and the system controller controls the multiplexer such that the plurality of pulses of energy from the energy source are sequentially directed to each of the plurality of energy guides in accordance with the firing sequence.

In some implementations, the plurality of energy guides includes a first energy guide, a second energy guide and a third energy guide that are each disposed along the catheter shaft and at least partially within the balloon interior, (i) the first energy guide including a first guide distal end that is positioned within the balloon interior at a first longitudinal position along the length of the balloon, (ii) the second energy guide including a second guide distal end that is positioned within the balloon interior at a second longitudinal position along the length of the balloon that is different than the first longitudinal position, and (iii) the third energy guide including a third guide distal end that is positioned within the balloon interior at a third longitudinal position along the length of the balloon that is different than the first longitudinal position and the second longitudinal position, the second longitudinal position being between the first longitudinal position and the third longitudinal position along the length of the balloon; and wherein the system controller controls the energy source such that that the plurality of pulses of energy from the energy source are sequentially directed to the first energy guide, then the second energy guide, and then the third energy guide so that the advancing wavefront is generated within the balloon interior that moves toward the treatment site in the first direction from near the balloon first end toward the balloon second end.

In certain implementations, the balloon first end is a balloon proximal end and the balloon second end is a balloon distal end; and the system controller controls the energy source such that the plurality of pulses of energy from the energy source are sequentially directed to the first energy guide, then the second energy guide, and then the third energy guide so that the advancing wavefront is generated within the balloon interior that moves toward the treatment site in the first direction from near the balloon proximal end toward the balloon distal end.

In other implementations, the balloon first end is a balloon distal end and the balloon second end is a balloon proximal end; and the system controller controls the energy source such that the plurality of pulses of energy from the energy source are sequentially directed to the first energy guide, then the second energy guide, and then the third energy guide so that the advancing wavefront is generated within the balloon interior that moves toward the treatment site in the first direction from near the balloon distal end toward the balloon proximal end.

In some embodiments, the plurality of energy guides further includes a fourth energy guide that is disposed along the catheter shaft and at least partially within the balloon interior, the fourth energy guide including a fourth guide distal end that is positioned within the balloon interior at a fourth longitudinal position along the length of the balloon that is different than the first longitudinal position, the second longitudinal position and the third longitudinal position, the third longitudinal position being between the second longitudinal position and the fourth longitudinal position along the length of the balloon.

In certain implementations, the system controller controls the energy source such that the plurality of pulses of energy from the energy source are sequentially directed to the first energy guide, then the second energy guide, then the third energy guide, and then the fourth energy guide so that the advancing wavefront is generated within the balloon interior that moves toward the treatment site in the first direction from near the balloon first end toward the balloon second end.

In some embodiments, the plurality of energy guides further includes a fifth energy guide that is disposed along the catheter shaft and at least partially within the balloon interior, the fifth energy guide including a fifth guide distal end that is positioned within the balloon interior at a fifth longitudinal position along the length of the balloon that is different than the first longitudinal position, the second longitudinal position, the third longitudinal position and the fourth longitudinal position, the fourth longitudinal position being between the third longitudinal position and the fifth longitudinal position along the length of the balloon.

In certain implementations, the system controller controls the energy source such that the plurality of pulses of energy from the energy source are sequentially directed to the first energy guide, then the second energy guide, then the third energy guide, then the fourth energy guide, and then the fifth energy guide so that the advancing wavefront is generated within the balloon interior that moves toward the treatment site in the first direction from near the balloon first end toward the balloon second end.

In other implementations, the system controller controls the energy source such that a first plurality of pulses of energy from the energy source are sequentially directed to the first energy guide, then the second energy guide, and then the third energy guide so that the advancing wavefront is generated within the balloon interior that moves toward the treatment site in the first direction from near the balloon first end toward the balloon second end; and the system controller controls the energy source such that a second plurality of pulses of energy from the energy source are sequentially directed to the fifth energy guide, then the fourth energy guide, and then the third energy guide so that a second advancing wavefront is generated within the balloon interior that moves toward the treatment site in a second direction from near the balloon second end toward the balloon first end.

In some embodiments, the advancing wavefront is spherical-shaped and impinges at an angle relative to the balloon wall to create a shearing force at the balloon wall substantially adjacent to the treatment site.

In various embodiments, each of the plurality of energy guides includes an optical fiber.

In many embodiments, the energy source is a laser source that generates a plurality of pulses of laser energy.

In other embodiments, the energy source generates a plurality of electrical impulses.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic cross-sectional view illustration of an embodiment of a catheter system in accordance with various embodiments herein, the catheter system including an energy source, a plurality of energy guides, a multiplexer, and a system controller that controls at least the energy source and the multiplexer;

FIG. 2A is a simplified schematic top view illustration of a portion of an embodiment of the catheter system including one embodiment of the multiplexer;

FIG. 2B is a simplified schematic perspective view illustration of a portion of the catheter system and the multiplexer illustrated in FIG. 2A;

FIG. 3A is a simplified schematic top view illustration of a portion of an embodiment of the catheter system including another embodiment of the multiplexer;

FIG. 3B is a simplified schematic perspective view illustration of a portion of the catheter system and the multiplexer illustrated in FIG. 3A;

FIG. 4 is a simplified schematic top view illustration of a portion of the catheter system and still another embodiment of the multiplexer;

FIG. 5 is a simplified schematic top view illustration of a portion of the catheter system and yet another embodiment of the multiplexer;

FIG. 6 is a simplified schematic top view illustration of a portion of the catheter system and another embodiment of the multiplexer;

FIG. 7 is a simplified schematic top view illustration of a portion of the catheter system and still another embodiment of the multiplexer;

FIG. 8A is a simplified schematic side view illustration of a portion of another embodiment of the catheter system;

FIG. 8B is a simplified schematic cross-sectional view illustration of the portion of the catheter system taken on line B-B in FIG. 8A;

FIG. 9 is a simplified schematic cross-sectional view illustration of another embodiment of the catheter system;

FIG. 10 is a simplified schematic cross-sectional view illustration of still another embodiment of the catheter system;

FIG. 11 is a simplified schematic cross-sectional view illustration of yet another embodiment of the catheter system;

FIG. 12 is a simplified schematic cross-sectional view illustration of another embodiment of the catheter system;

FIG. 13 is a simplified schematic cross-sectional view illustration of still another embodiment of the catheter system; and

FIGS. 14A-14H are graphical representations of combinations of pulse frequency and pulse energy level that can be controlled by the system controller for purposes of generating desired alternative types of advancing wavefronts.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions at treatment sites within a body of a patient can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs. In certain implementations, a “treatment site” can be located at or near a vessel wall of a blood vessel of the patient. Additionally, or in the alternative, in other implementations, a “treatment site” can be at or near a heart valve of the patient. Further, or in the alternative, in still other implementations, a “treatment site” can be at another suitable location within the body of the patient.

It is noted that the terms “intravascular lesion”, “vascular lesion” and “treatment site” are used interchangeably herein unless otherwise noted. The intravascular lesions and/or the vascular lesions are sometimes referred to herein simply as “lesions”, and can be located at or near a vessel wall of a blood vessel, at or near a heart valve of the patient, and/or at any other suitable location (or treatment site) within the body of the patient.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

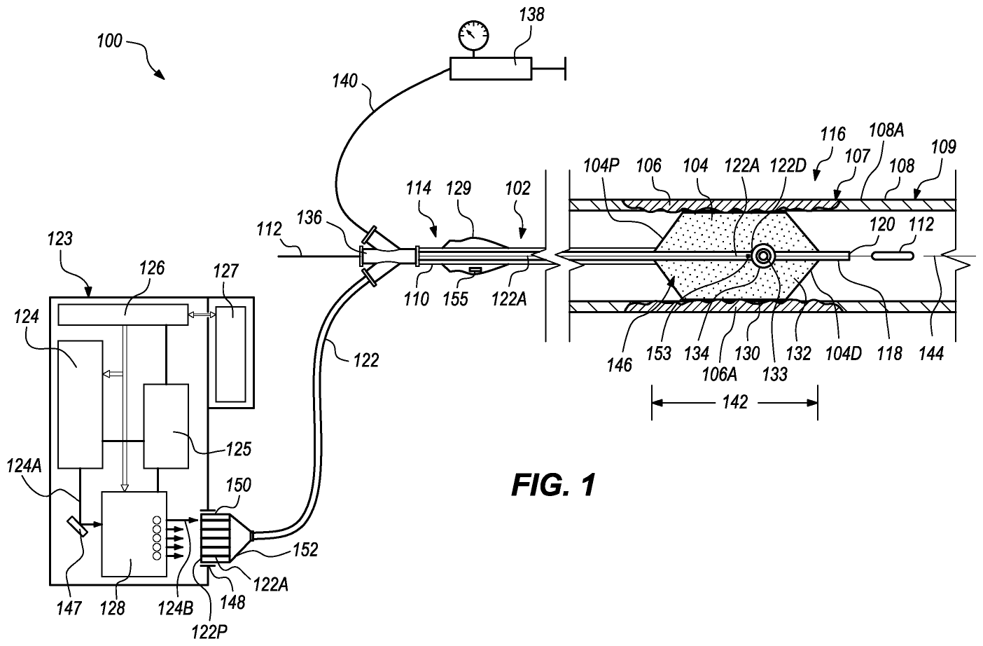

The catheter systems disclosed herein can include many different forms. Referring now to FIG. 1, a simplified schematic cross-sectional view illustration is shown of a catheter system 100 in accordance with various embodiments. The catheter system 100 is suitable for imparting pressure waves to induce fractures in one or more vascular lesions 106A at a treatment site 106 within or adjacent to a vessel wall 108A of a blood vessel 108, or on or adjacent to a heart valve, within a body 107 of a patient 109. In the embodiment illustrated in FIG. 1, the catheter system 100 can include (i) a catheter 102 including one or more of an inflatable balloon 104 (sometimes referred to herein as a “balloon”) including a balloon wall 130 that defines a balloon interior 146, a catheter shaft 110, a guidewire 112, an energy guide bundle 122 including a plurality of energy guides 122A, a source manifold 136, a fluid pump 138, and a handle assembly 129, and (ii) a system console 123 including one or more of an energy source 124, a power source 125, a system controller 126, a graphic user interface 127 (a “GUI”), and a multiplexer 128. Alternatively, the catheter system 100, the catheter 102 and/or the system console 123 can include more components or fewer components than those specifically illustrated and described in relation to FIG. 1.

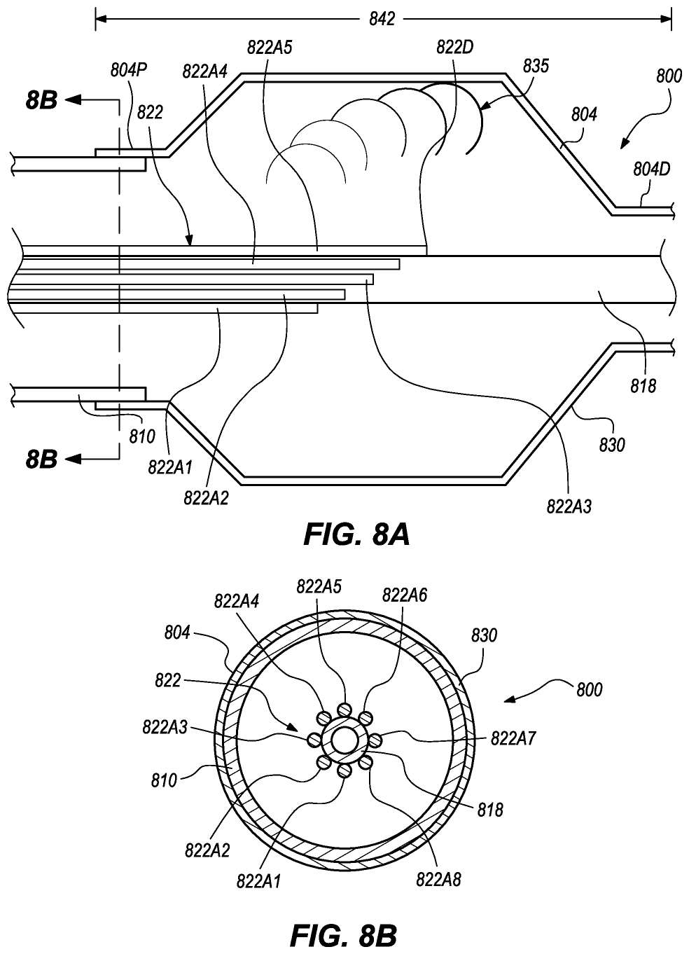

As an overview, in various embodiments, the present invention is directed toward a catheter system 100 in the form of an intravascular lithotripsy device that generates an advancing energy wavefront (also referred to as an “advancing wavefront”) in order to effectively treat the vascular lesions 106A, such as calcified vascular lesions or fibrous vascular lesions, at the treatment site 106 within the body 107 of the patient 109. An exemplary, representative advancing wavefront 835 is illustrated in FIG. 8A. More particularly, the intravascular lithotripsy device, or catheter system 100, can include the energy source 124 that generates energy; the plurality of energy guides 122A that are positioned in a desired manner relative to the treatment site 106 along a length 142 of the balloon 104 and that are each configured to selectively receive the energy from the energy source 124; and the system controller 126 that controls the energy source 124 so that pulses of energy from the energy source 124 are directed to each of the plurality of energy guides 122A in a desired sequence, with a desired pulse frequency, and with desired energy parameters, so that the advancing wavefront 835 is generated that moves toward the treatment site 106. In certain embodiments, the catheter system 100 can further include the multiplexer 128 that is controlled by the system controller 126 so that a single energy source 124 can be utilized to generate a single pulsed source beam 124A that is directed toward and/or through the multiplexer 128 to produce a plurality of individual guide beams 124B that are directed to each of the plurality of energy guides 122A in the desired sequence, with the desired pulse frequency, and with the desired energy parameters.

As used herein, the term “advancing wavefront” is intended to signify the resulting wavefront produced by a series of guide beams 124B that are directed to the plurality of energy guides 122A so that an overall pattern is generated wherein light energy causing pressure waves is generally moving toward the treatment site 106. More particularly, in various implementations, the “advancing wavefront” encompasses acoustic energy that is directed at least in part either forward (distally) or backward (proximally), or both, relative to the length 142 of the balloon 104, and thus includes more than just radial energy that is directed sideways toward the balloon wall 130.

Conversely, a “retreating wavefront” would in effect be somewhat the opposite of an advancing wavefront. In other words, a retreating wavefront would be a wavefront produced by a series of guide beams 124B that are directed to the plurality of energy guides 122A so that an overall pattern is generated wherein light energy causing pressure waves are generally moving away from the treatment site 106. It is understood that those skilled in the art would understand the retreating wavefront to operate in substantially the opposite manner as the advancing wavefront.

In many embodiments, the advancing wavefront 835 is spherical-shaped and impinges at an angle (non-normal) relative to the balloon wall 130 to create a shearing force at the balloon wall 130 substantially adjacent to the treatment site 106. More particularly, in several embodiments, the system controller 126 can control the energy source 124 and/or the multiplexer 128 to create an advancing series of energy waves along the length 142 of the balloon 104 for creating a shear wave in the vascular lesion(s) 106A at the treatment site 106. In such applications, as individual guide beams 124B are directed to specific energy guides 122A in a specific sequence, with desired pulse frequency, and with desired energy parameters, the pressure waves will advance in the direction of activation. As new bubbles are created ahead of collapsing ones, it would be possible to create a shearing force at the balloon wall 130. The localized force on the leading edge of the spherical wavefront impinging at an angle relative to the balloon wall 130 that is non-normal creates a highly concentrated, localized shearing force. This could have a greater effect in cracking calcified lesions compared to simply hitting the walls through the length 142 of the balloon 104 with one radially directed pressure wave, which can expand the whole cross-section of the balloon 104 creating hoop stress. Thus, as described in detail herein, in various embodiments, the present invention can be utilized to solve various problems that exist in more traditional catheter systems.

It is appreciated that the catheter system 100 having features of the present invention can be utilized to generate various different types of advancing wavefronts 835 by manipulating energy parameters in different ways. It is further appreciated that different types of advancing wavefronts 835 can be more suitable for different types, sizes (thickness, length, etc.) and shapes of vascular lesions 106A.

For example, in order to generate different types of advancing wavefronts 835, the system controller 126 can control the energy source 124 to generate pulses of energy that can vary in terms of such parameters as pulse frequency, pulse energy level, and/or pulse width (or pulse duration). More specifically, the pulse frequency of successive energy pulses generated by the energy source 124 can be controlled by the system controller 126 to be increasing, decreasing and/or constant as successive energy guides 122A are activated (such as by the multiplexer 128 directing individual energy beams 124B to the individual energy guides 122A in the desired firing sequence). Similarly, the pulse energy level of successive energy pulses generated by the energy source 124 can also be controlled by the system controller 126 to be increasing, decreasing and/or constant as successive energy guides 122A are activated (such as by the multiplexer 128 directing individual energy beams 124B to the individual energy guides 122A in the desired firing sequence). Still similarly, the pulse width of successive energy pulses generated by the energy source 124 can further be controlled by the system controller 126 to be increasing, decreasing and/or constant as successive energy guides 122A are activated (such as by the multiplexer 128 directing individual energy beams 124B to the individual energy guides 122A in the desired firing sequence).

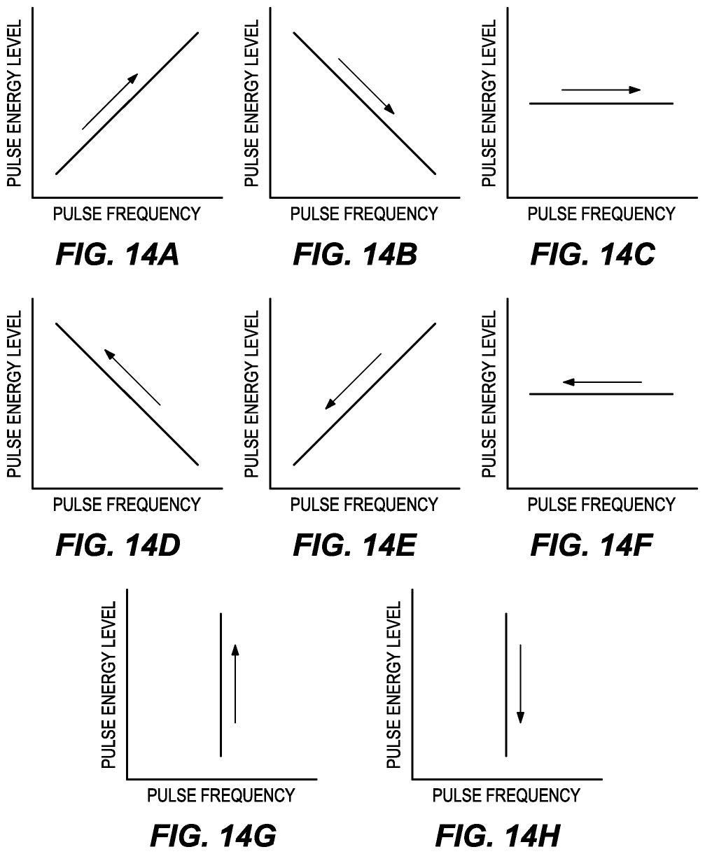

As such, different types of advancing wavefronts 835 can be generated by using a combination of (i) increasing pulse frequency and increasing pulse energy level; (ii) increasing pulse frequency and decreasing pulse energy level; (iii) increasing pulse frequency and constant pulse energy level; (iv) decreasing pulse frequency and increasing pulse energy level; (v) decreasing pulse frequency and decreasing pulse energy level; (vi) decreasing pulse frequency and constant pulse energy level; (vii) constant pulse frequency and increasing pulse energy level; (viii) constant pulse frequency and decreasing pulse energy level; and/or (ix) constant pulse frequency and constant pulse energy level. Representative examples of combinations (i) through (viii) are shown graphically in FIGS. 14A-14H. The types of advancing wavefronts 835 can further be modified from the noted combinations by using pulse widths that are either increasing, decreasing and/or constant with any of such combinations.

In a pressure wave generating medical device such as described herein, it is often desirable to have a number of potential output channels for the treatment process. For safety and convenience, these output channels can consist of optical fibers. Since a high-power laser source is often the largest and most expensive component in the system, it can be advantageous to utilize a single laser source that can be multiplexed into a number of different optical fibers for treatment purposes. This allows the possibility for using all of the laser power with each optical fiber. However, although the present invention is often described herein as using a single laser source for purposes of generating the desired pressure waves, it is appreciated that the present invention is not limited to the use of a laser-generated pressure wave system. For example, the present invention can alternatively use any suitable type of device that utilizes a highly localized energy source to generate the desired pressure waves. In one non-exclusive alternative example, the energy source can generate electrical impulses that are directed through the energy guides to generate the desired pressure waves, and the output channels, or energy guides, can be electrical conductors. It is appreciated that the present invention can also utilize more than one laser source and/or more than one other suitable pressure wave generating device.

As noted, in various embodiments, the catheter systems and related methods disclosed herein are configured to provide a means to power multiple fiber optic channels in a pressure wave generating device that is designed to impart pressure onto and induce fractures in vascular lesions using a single energy source. As described in detail herein, in certain embodiments, the catheter systems can be configured and controlled to selectively and/or separately power the multiple fiber optic channels in any desired firing sequence, pattern, order, firing rate and/or firing duration, etc. Thus, the invention described in detail herein can include a single energy source, which can be multiplexed into one or more of a plurality of energy guides in a single use device. This allows a single, stable energy source to be channeled sequentially in any desired firing sequence and at any firing rate and/or duration through any or all of the plurality of energy guides.

During use of the catheter system 100, the catheter 102 is configured to move to the treatment site 106 within or adjacent to the vessel wall 108A of the blood vessel 108 within the body 107 of the patient 109. The treatment site 106 can include one or more vascular lesions 106A such as calcified vascular lesions, for example. Additionally, or in the alternative, the treatment site 106 can include vascular lesions 106A such as fibrous vascular lesions. Still alternatively, in some implementations, the catheter 102 can be used at a treatment site 106 within or adjacent to a heart valve within the body 107 of the patient 109.

In certain embodiments, the balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter 102 and/or the catheter shaft 110 can also include a guidewire lumen 118 which is configured to move over the guidewire 112 so that the catheter 102 is accurately guided to the treatment site 106. As utilized herein, the guidewire lumen 118 defines a conduit through which the guidewire 112 extends. The catheter shaft 110 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106. In certain embodiments, the balloon proximal end 104P can be coupled to the catheter shaft 110, and the balloon distal end 104D can be coupled to the guidewire lumen 118.

It is appreciated that the balloon proximal end 104P and the balloon distal end 104D can each also be referred to as a “balloon first end” and/or a “balloon second end”. More specifically, in certain implementations, the balloon proximal end 104P can be referred to as the “balloon first end” and the balloon distal end 104D can be referred to as the “balloon second end”; and, in other implementations, the balloon distal end 104D can be referred to as the “balloon first end” and the balloon proximal end 104P can be referred to as the “balloon second end”.

The balloon 104 includes the balloon wall 130 that defines the balloon interior 146. The balloon 104 can be selectively inflated with a catheter fluid 132 to expand from a deflated state suitable for advancing the catheter 102 through a patient's vasculature, to an inflated state (as shown in FIG. 1) suitable for anchoring the catheter 102 in position relative to the treatment site 106. Stated in another manner, when the balloon 104 is in the inflated state, the balloon wall 130 of the balloon 104 is configured to be positioned substantially directly adjacent to and/or in contact with the treatment site 106.

The balloon 104 suitable for use in the catheter system 100 includes those that can be passed through the vasculature of a patient 109 when in the deflated state. In some embodiments, the balloon 104 is made from silicone. In other embodiments, the balloon 104 can be made from polydimethylsiloxane (PDMS), polyurethane, polymers such as PEBAX™ material, nylon, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In certain embodiments, the balloon 104 can have a diameter (in the inflated state) ranging from at least two mm up to five mm.

In some embodiments, the balloon 104 can have a length 142 ranging from at least three mm to 300 mm. More particularly, in certain embodiments, the balloon 104 can have a length 142 ranging from at least eight mm to 200 mm. It is appreciated that a balloon 104 having a relatively longer length can be positioned adjacent to larger treatment sites 106, and, thus, may be usable for imparting pressure waves and/or advancing wavefronts 835 onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloon 104 can include a drug eluting coating or a drug eluting stent structure. The drug eluting coating or drug eluting stent can include one or more therapeutic agents including anti-inflammatory agents, anti-neoplastic agents, anti-angiogenic agents, and the like.

The catheter fluid 132 used to inflate the balloon 104 can be a liquid or a gas. Some examples of the catheter fluid 132 suitable for use can include, but are not limited to, one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the pressure waves are appropriately manipulated. In certain embodiments, the catheter fluid 132 suitable for use herein is biocompatible. A volume of catheter fluid 132 can be tailored by the chosen energy source 124 and the type of catheter fluid 132 used.

In some embodiments, the contrast agents used in the contrast media can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine-based contrast agents can be used. Suitable non-iodine containing contrast agents can include gadolinium (III)-based contrast agents. Suitable fluorocarbon and perfluorocarbon agents can include, but are not to be limited to, agents such as perfluorocarbon dodecafluoropentane (DDFP, C5F12).

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium:yttrium-aluminum-garnet (Nd:YAG−emission maximum=1064 nm) lasers, holmium: YAG (Ho:YAG−emission maximum=2.1 μm) lasers, or erbium: YAG (Er: YAG−emission maximum=2.94 μm) lasers. In some embodiments, the absorptive agents can be water soluble. In other embodiments, the absorptive agents are not water soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

In various embodiments, the catheter shaft 110 can be coupled to the plurality of energy guides 122A of the energy guide bundle 122 that can be in optical and/or electrical communication with the energy source 124. The energy guide(s) 122A can be disposed along the catheter shaft 110 and within the balloon 104. Each of the energy guides 122A can have a guide distal end 122D that is at any suitable longitudinal position relative to the length 142 of the balloon 104 and/or relative to a length of the guidewire lumen 118. As provided herein, the guide distal end 122D of each of the plurality of energy guides 122D can be positioned in any suitable locations relative to the length 142 of the balloon 104 to more effectively and precisely impart pressure waves for purposes of disrupting the vascular lesions 106A at the treatment site 106. Further, with the configuration of the present invention, it is possible to fire individual energy guides 122A, including one or more energy guides 122A that are fired substantially simultaneously or sequentially, to achieve a firing sequence or pattern that could be more effective at disrupting localized lesions 106A. Firing separate plasma generator channels in a predetermined and/or specific firing sequence or pattern, with desired energy parameters, can create the advancing wavefront 835 that more effectively breaks up a lesion 106A in one location or an extended lesion 106A.

It is further appreciated that the longitudinal distance (relative to the length 142 of the balloon 104) between the guide distal ends 122D of successive energy guides 122A within a given firing sequence can further impact the generation of the advancing wavefront 835.

Thus, as taught herein, the type of advancing wavefront 835 and manner for generating the advancing wavefront 835 can be influenced at least in part by one or more of (i) the firing sequence of successive energy guides, (ii) the pulse frequency of the energy pulses, (iii) the pulse energy level of the energy pulses, (iv) the pulse width (or pulse duration) of the energy pulses, and (v) the longitudinal spacing between the guide distal end 122D of successive energy guides 122A within the firing sequence. It is further contemplated that the type of advancing wavefront 835 and manner for generating the advancing wavefront 835 can also be influenced by other energy parameters. Moreover, it is also appreciated that the advancing wavefront 835 can be generated through a firing sequence of any two or more energy guides 122A, although the number of energy guides 122A incorporated within a given firing sequence will also impact the specific type of advancing wavefront 835 that is generated.

It is further appreciated that, depending on the specific positioning of the balloon 104 relative to the vascular lesions 106A at the treatment site 106, and/or the specific energy guides 122A used for generating the advancing wavefront 835 (and the positioning thereof), the generated advancing wavefront 835 can be said to move toward the treatment site 106 in a direction from near the balloon proximal end 104P toward the balloon distal end 104D, from near the balloon distal end 104D toward the balloon proximal end 104P, or more generally from near the balloon first end toward the balloon second end.

In some embodiments, each energy guide 122A can be an optical fiber and the energy source 124 can be a laser. The energy source 124 can be in optical and/or electrical communication with the energy guides 122A at the proximal portion 114 of the catheter system 100. More particularly, the energy source 124 can selectively, simultaneously, sequentially and/or alternatively be in optical and/or electrical communication with each of the energy guides 122A in any desired combination, sequence and/or pattern due to the presence and operation of the multiplexer 128. Alternatively, each energy guide 122A can have another suitable design and/or the energy source 124 can be another suitable energy source.

In some embodiments, the catheter shaft 110 can be coupled to multiple energy guides 122A such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart from one another by approximately 180 degrees about the circumference of the guidewire lumen 118 and/or the catheter shaft 110; three energy guides 122A can be spaced apart from one another by approximately 120 degrees about the circumference of the guidewire lumen 118 and/or the catheter shaft 110; four energy guides 122A can be spaced apart from one another by approximately 90 degrees about the circumference of the guidewire lumen 118 and/or the catheter shaft 110; five energy guides 122A can be spaced apart from one another by approximately 72 degrees about the circumference of the guidewire lumen 118 and/or the catheter shaft 110; six energy guides 122A can be spaced apart from one another by approximately 60 degrees about the circumference of the guidewire lumen 118 and/or the catheter shaft 110; eight energy guides 122A can be spaced apart from one another by approximately 45 degrees about the circumference of the guidewire lumen 118 and/or the catheter shaft 110; or ten energy guides 122A can be spaced apart from one another by approximately 36 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations. In certain embodiments, the guidewire lumen 118 can have a grooved outer surface, with the grooves extending in a generally longitudinal direction along the guidewire lumen 118. In such embodiments, each of the energy guides 122A can be positioned, received, and retained within an individual groove formed along and/or into the outer surface of the guidewire lumen 118. Alternatively, the guidewire lumen 118 can be formed without a grooved outer surface, and the position of the energy guides 122A relative to the guidewire lumen 118 can be maintained in another suitable manner. The catheter system 100, the catheter 102 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100, the catheter 102 and/or the energy guide bundle 122 can include from one energy guide 122A to greater than 30 energy guides 122A. Alternatively, in other embodiments, the catheter system 100, the catheter 102 and/or the energy guide bundle 122 can include greater than 30 energy guides 122A.

The energy guides 122A can be disposed at any suitable positions about the circumference of the guidewire lumen 118 and/or the catheter shaft 110, and the guide distal end 122D of each of the energy guides 122A can be disposed at any suitable longitudinal position relative to the length 142 of the balloon 104 and/or relative to the length of the guidewire lumen 118 to more effectively and more precisely impart pressure waves for purposes of disrupting the vascular lesions 106A at the treatment site 106. At least a portion of one or more of the energy guides 122A can be positioned spaced apart from the guidewire lumen 118, such that the guide distal end of such energy guides 122A can be positioned at any suitable position laterally between the guidewire lumen 118 and the balloon wall 130 of the balloon 104.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

In various embodiments, the energy guides 122A can each include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P toward the guide distal end 122D, with the guide distal end 122D having at least one optical window (not shown) that is positioned within the balloon interior 146.

In certain embodiments, the energy guides 122A can include one or more photoacoustic transducers 153, where each photoacoustic transducer 153 can be in optical communication with the energy guide 122A within which it is disposed. In some embodiments, the photoacoustic transducers 153 can be in optical communication with the guide distal end 122D of the energy guide 122A. In such embodiments, the photoacoustic transducers 14 can have a shape that corresponds with and/or conforms to the guide distal end 122D of the energy guide 122A.

The photoacoustic transducer 153 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 153 disposed at the guide distal end 122D of the energy guide 122A can assume the same shape as the guide distal end 122D of the energy guide 122A. For example, in certain non-exclusive embodiments, the photoacoustic transducer 153 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 153 disposed along one or more side surfaces of the length of the energy guide 122A.

In some embodiments, the energy guides 122A can further include one or more diverting structures or “diverters” (not shown in FIG. 1), such as within the energy guide 122A and/or near the guide distal end 122D of the energy guide 122A, that are configured to direct energy from the energy guide 122A toward a side surface which can be located at or near the guide distal end 122D of the energy guide 122A, before the energy is directed toward the balloon wall 130. A diverting structure can include any structure of the system that diverts energy from the energy guide 122A away from its axial path toward a side surface of the energy guide 122A. The energy guides 122A can each include one or more optical windows disposed along the longitudinal or circumferential surfaces of each energy guide 122A and in optical communication with a diverting structure. Stated in another manner, the diverting structures can have any suitable structural configuration that is configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting structures suitable for use include a reflecting element, a refracting element, and a fiber diffuser. The diverting structures suitable for focusing energy away from the tip of the energy guides 122A can include, but are not to be limited to, those having a convex surface, a gradient-index (GRIN) lens, and a mirror focus lens. Upon contact with the diverting structure, the energy can be diverted within the energy guide 122A to one or more of a plasma generating structure 133 (also sometimes referred to as a “plasma generator” or “plasma target”) that is positioned near, but typically spaced apart from, the guide distal end 122D of the energy guide 122A, and the photoacoustic transducer 153 that is in optical communication with a side surface of the energy guide 122A. As referred to herein, the plasma generator 133 can include and/or incorporate any suitable type of structure that is located at or near the guide distal end 122D of the energy guide 122A.

When utilized, the plasma generator 133 receives energy emitted from the guide distal end 122D of the energy guide 122A to generate plasma in the catheter fluid 132 within the balloon interior 146, which, in turn, causes the creation of plasma bubbles 134 and/or pressure waves that can be directed away from the side surface of the energy guide 122A and toward the balloon wall 130. Additionally, or in the alternative, when utilized, the photoacoustic transducer 153 converts light energy into an acoustic wave that extends away from the side surface of the energy guide 122A.

As provided herein, the catheter system 100 can utilize light energy from the energy source 124, such as a laser source or other suitable energy source, to generate a plasma within the catheter fluid 132 at or near the guide distal end 122D of each of the plurality of energy guides 122A disposed in the balloon 104 located at the treatment site 106. The plasma formation can initiate one or more pressure waves by initiating rapid formation of one or more bubbles that can rapidly expand to a maximum size and then dissipate through a cavitation event that can launch a pressure wave upon collapse. Alternatively, the plasma formation can initiate an explosive type of pressure wave or pressure waves that can extend to the treatment site 106 to disrupt calcification. Stated another way, the rapid expansion of the plasma-induced bubbles 134 can generate one or more pressure waves within the balloon fluid 132 retained within the balloon 104 and thereby impart pressure waves upon the treatment site 106.

Importantly, as noted, the system controller 126 controls the energy source 124 to generate pulses of energy for purposes of firing any two or more of the plurality of energy guides 122A in a desired firing sequence, with a desired pulse frequency, with a desired pulse energy level, with a desired pulse width (or pulse duration), and with the guide distal end 122D of successive energy guides 122A (within the desired firing sequence) being longitudinally positioned relative to one another with a desired spacing relative to the length 142 of the balloon 104. By controlling any or all of these parameters, the catheter system 100 can be effectively utilized to generate any desired type of advancing wavefront 835 for purposes of breaking the vascular lesions 106A at the treatment site 106.

In various embodiments, the advancing wavefront 835 can be generated using any desired number of energy guides 122A. For example, in certain non-exclusive alternative embodiments, the advancing wavefront 835 can be generated using from two energy guides 122A to ten energy guides 122A in a desired firing sequence. More particularly, in such embodiments, the advancing wavefront 835 can be generated using two, three, four, five, six, seven, eight, nine or ten energy guides 122A in a desired firing sequence. Alternatively, the advancing wavefront 835 can be generated using more than ten energy guides 122A in a desired firing sequence.

The time between successive pulses (inverse of the pulse frequency) can be varied as desired to generate the desired type of advancing wavefront 835 for purposes of breaking the vascular lesions 106A at the treatment site 106. In some non-exclusive embodiments, the time between successive pulses can be between approximately 50 milliseconds (ms) and five seconds. In other embodiments, the time between successive pulses can be between approximately 75 ms and one second. In still other embodiments, the time between successive pulses can be between approximately 100 ms and 500 ms. Alternatively, the time between successive pulses can be less than 50 ms, greater than five seconds, and/or within another suitable range. For example, in certain non-exclusive alternative embodiments, the time between successive pulses can be less than one ms, such as between approximately one microsecond and ten microseconds, between approximately ten microseconds and 100 microseconds, and/or between approximately 100 microseconds and one millisecond. It is further appreciated that in order to generate the desired type of advancing wavefront 835, the time between successive pulses can be increasing, decreasing or substantially constant during the desired firing sequence.

The pulse energy level (for individual energy pulses) can also be varied as desired to generate the desired type of advancing wavefront 835 for purposes of breaking the vascular lesions 106A at the treatment site 106. In certain non-exclusive embodiments, the pulse energy level can be between approximately one millijoule (mJ) and 60 mJ. In other embodiments, the pulse energy level can be between approximately two mJ and 40 mJ. In still other embodiments, the pulse energy level can be between approximately five mJ and 25 mJ. Alternatively, the pulse energy level (for individual energy pulses) can be less than one mJ, greater than 60 mJ, and/or within another suitable range. It is further appreciated, as noted above, that in order to generate the desired type of advancing wavefront 835, the pulse energy level can be increasing, decreasing or substantially constant during the desired firing sequence.

The pulse width (or pulse duration) can also be varied as desired to generate the desired type of advancing wavefront 835 for purposes of breaking the vascular lesions 106A at the treatment site 106. In some non-exclusive alternative embodiments, the pulse width can be between approximately 100 nanoseconds (ns) and 1600 ns. In other embodiments, the pulse width can be between approximately 200 ns and 1200 ns. In still other embodiments, the pulse width can be between approximately 250 ns and 1000 ns. Alternatively, the pulse width (or pulse duration) can be less than 100 ns, greater than 1600 ns, and/or within another suitable range. It is further appreciated, as noted above, that in order to generate the desired type of advancing wavefront 835, the pulse width can be increasing, decreasing or substantially constant during the desired firing sequence.

The longitudinal spacing (relative to the length 142 of the balloon 104) between the guide distal end 122D of successive energy guides 122A utilized within the desired firing sequence can be varied as desired to generate the desired type of advancing wavefront 835 for purposes of breaking the vascular lesions 106A at the treatment site 106. In certain non-exclusive alternative embodiments, the longitudinal spacing between the guide distal end 122D of successive energy guides 122A utilized within the desired firing sequence can be between approximately one millimeter (mm) and 100 mm. In other embodiments, the longitudinal spacing between the guide distal end 122D of successive energy guides 122A utilized within the desired firing sequence can be between approximately five mm and 75 mm. In still other embodiments, the longitudinal spacing between the guide distal end 122D of successive energy guides 122A utilized within the desired firing sequence can be between approximately ten mm and 50 mm. Alternatively, the longitudinal spacing between the guide distal end 122D of successive energy guides 122A utilized within the desired firing sequence can be less than one mm, greater than 100 mm, and/or within another suitable range. It is further appreciated that in order to generate the desired type of advancing wavefront 835, the longitudinal spacing between the guide distal end 122D of successive energy guides 122A utilized within the desired firing sequence can be increasing, decreasing or substantially constant during the desired firing sequence.

The source manifold 136 can be positioned at or near the proximal portion 114 of the catheter system 100. The source manifold 136 can include one or more proximal end openings that can receive the plurality of energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the catheter fluid 132 as needed.

As provided herein, the system console 123 includes one or more of the energy source 124, the power source 125, the system controller 126, the GUI 127, and the multiplexer 128. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, the GUI 127 and the multiplexer 128 can be provided within the catheter system 100 without the specific need for the system console 123.

As shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (which can generally include one or more ferrules) that houses a portion, such as the guide proximal end 122P, of each of the energy guides 122A. At least a portion of the guide coupling housing 150 is configured to fit and be selectively retained within the console connection aperture 148 to provide the desired mechanical coupling between the energy guide bundle 122 and the system console 123, as well as helping to provide an optical coupling between the energy source 124 and the energy guides 122A of the energy guide bundle 122.

The energy guide bundle 122 can also include a guide bundler 152 (or “shell”) that brings each of the individual energy guides 122A closer together so that the energy guides 122A and/or the energy guide bundle 122 can be in a more compact form as it extends as part of the catheter 102 into the blood vessel 108 during use of the catheter system 100.

The energy source 124 can be selectively and/or alternatively coupled in optical communication with each of the energy guides 122A, such as to the guide proximal end 122P of each of the energy guides 122A, in the energy guide bundle 122. In particular, the energy source 124 is configured to generate light energy in the form of a source beam 124A, such as a pulsed source beam, that can be selectively and/or alternatively directed to and received by each of the energy guides 122A in the energy guide bundle 122 as an individual guide beam 124B. More specifically, as described in greater detail herein below, the source beam 124A from the energy source 124 is directed through the multiplexer 128 such that the individual guide beams 124B (or “multiplexed beams”) can be selectively and/or alternatively directed into and received by each of the energy guides 122A in the energy guide bundle 122. In particular, each pulse of the energy source 124, or each pulse of the source beam 124A, can be directed through the multiplexer 128 to generate a separate guide beam 124B that is selectively and/or alternatively directed onto one of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104, such as via the plasma generator 133 that can include and/or incorporate a structure that is located at or near the guide distal end 122D of the energy guide 122A. In many embodiments, the plasma generator 133 can be positioned slightly spaced apart from the guide distal end 122D of the energy guide 122A. In certain embodiments, the plasma generator 133 can be provided in the form of a backstop-type structure with an angled face that redirects energy emitted from the guide distal end 122D toward the balloon wall 130 of the balloon 104 and/or toward the vessel wall 108A of the blood vessel 108 at the treatment site 106.

In particular, the energy emitted at the guide distal end 122D of the energy guide 122A is directed toward and impinges on and energizes material of the plasma generator 133, such as material on an angled face of the plasma generator 133, for purposes of generating plasma in the catheter fluid 132 within the balloon interior 146. The plasma generation ionizes and superheats the surrounding catheter fluid 132 and thus causes rapid inertial bubble formation, and imparts pressure waves upon the treatment site 106. An exemplary plasma-induced bubble 134 is illustrated in FIG. 1.

The plasma generator 133 can be formed from any suitable materials. For example, in certain non-exclusive embodiments, the plasma generator 133 can be formed from one or more metals such as titanium, stainless steel, tungsten, tantalum, platinum, molybdenum, niobium, iridium, etc. Alternatively, the plasma generator 133 may be formed from plastics such as polyimide and nylon. Still alternatively, the plasma generator 133 can be formed from other suitable materials.

In various non-exclusive alternative embodiments, the sub-millisecond pulses of energy from the energy source 124 can be delivered to the treatment site 106 at a pulse frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of energy can be delivered to the treatment site 106 at a pulse frequency that can be greater than 5000 Hz or less than one Hz, or any other suitable range of pulse frequencies. It is further appreciated that in order to generate the desired type of advancing wavefront 835, the pulse frequency can be increasing, decreasing or substantially constant during the desired firing sequence.

It is appreciated that although the energy source 124 is typically utilized to provide pulses of energy, the energy source 124 can still be described as providing a single source beam 124A, i.e. a single pulsed source beam.

The energy source 124 suitable for use can include various types of energy sources including, but not limited to, lasers and lamps. For example, in certain non-exclusive embodiments, the energy source 124 can be an infrared laser that emits energy in the form of pulses of infrared light. Alternatively, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (us) timescales. It is appreciated that there are many combinations of laser pulse wavelengths, pulse widths and amplitudes that provide varying energy levels that can be employed to achieve plasma in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

In certain specific embodiments, as noted above, the pulse width from a suitable laser could vary between 500 ns to 1600 ns. A shorter pulse tends to give higher acoustic energy for the same net input energy. In some implementations, a given pressure wave could start with a long pulse out at 1600 ns with set energy, then transition through steps along the desired firing sequence to shorter pulses.

Exemplary nanosecond lasers can include those within the UV to IR spectrum, spanning pulse wavelengths of about ten nanometers (nm) to one millimeter (mm). In some embodiments, the energy sources 124 suitable for use in the catheter systems 100 can include those capable of producing light at pulse wavelengths of from at least 750 nm to 2000 nm. In other embodiments, the energy sources 124 can include those capable of producing light at pulse wavelengths of from at least 700 nm to 3000 nm. In still other embodiments, the energy sources 124 can include those capable of producing light at pulse wavelengths of from at least 100 nm to ten micrometers (μm). Nanosecond lasers can include those having repetition rates of up to 200 KHz.

In some embodiments, the laser can include a Q-switched thulium:yttrium-aluminum-garnet (Tm:YAG) laser. In other embodiments, the laser can include a neodymium: yttrium-aluminum-garnet (Nd: YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 (or cavitation bubble) in the catheter fluid 132.

The catheter system 100 can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In various non-exclusive alternative embodiments, the catheter system 100 can generate pressure waves having maximum pressures in the range of at least approximately two MPa to 50 MPa, at least approximately two MPa to 30 MPa, or at least approximately 15 MPa to 25 MPa.

The pressure waves can be imparted upon the treatment site 106 from a distance within a range from at least approximately 0.1 millimeters (mm) to greater than approximately 25 mm extending radially from the energy guides 122A when the catheter 102 is placed at the treatment site 106. In various non-exclusive alternative embodiments, the pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the pressure waves can be imparted upon the treatment site 106 from another suitable distance that is different than the foregoing ranges. In some embodiments, the pressure waves can be imparted upon the treatment site 106 within a range of at least approximately two MPa to 30 MPa at a distance from at least approximately 0.1 mm to ten mm. In some embodiments, the pressure waves can be imparted upon the treatment site 106 from a range of at least approximately two MPa to 25 MPa at a distance from at least approximately 0.1 mm to ten mm. Still alternatively, other suitable pressure ranges and distances can be used.

The power source 125 is electrically coupled to and is configured to provide necessary power to one or more of the energy source 124, the system controller 126, the GUI 127, the multiplexer 128, and the handle assembly 129. The power source 125 can have any suitable design for such purposes.

The system controller 126 is electrically coupled to and receives power from the power source 125. The system controller 126 is coupled to and is configured to control operation of each of the energy source 124, the GUI 127 and the multiplexer 128. The system controller 126 can include one or more processors or circuits for purposes of controlling the operation of at least the energy source 124, the GUI 127 and the multiplexer 128. For example, as described in detail herein above, the system controller 126 can control the energy source 124 for generating pulses of energy as desired and/or at any desired firing rate or desired frequency of pulse delivery, at any desired pulse energy level, and/or at any desired pulse width (or pulse duration). Subsequently, the system controller 126 can then control the multiplexer 128 so that the light energy from the energy source 124, such as the source beam 124A, can be selectively and/or alternatively directed to each of the energy guides 122A, such as in the form of individual guide beams 124B, in a desired firing sequence.

More specifically, the system controller 126 can control the energy source 124 and/or the multiplexer 128 so that individual guide beams 124B can be directed to each of the energy guides 122A, or sets or subsets of the energy guides 122A, in a desired firing sequence, firing pattern, firing order, firing energy levels (which can any or all of include pulse width, pulse amplitude and/or pulse wavelength) and/or firing rate. For example, in a catheter system 100 that includes eight energy guides 122A, such as shown in FIGS. 8A and 8B, that are arranged in a linear pattern with angular orientation spiraling around the guidewire lumen 118, the system controller 126 can control the sequencing of the firing of the light energy from the energy source 124 to each of the energy guides 122A in any desired manner. As used herein, the term “firing rate” is intended to mean the number of pulses per a given time frame. Further, as used herein, the term “firing energy level” is intended to mean the intensity of the energy pulse, which can be varied depending upon the pulse width and/or the pulse amplitude of any or all of the pulse(s).

Certain non-exclusive examples of alternative applications of sequencing of the firing of the eight energy guides 122A will be described in greater detail herein. As used herein, different “desired firing sequences” can equally be referred to as a first firing sequence, a second firing sequence, a third firing sequence, etc. for ease of discussion and understanding. Somewhat similarly, different firing patterns, firing orders, firing energy levels and/or firing rates can likewise equally be referred to herein as a first, a second, a third, etc. for ease of discussion and understanding.

It is appreciated that although each of the plurality of energy guides can be powered separately in any desired firing sequence, pattern, order, firing rate, and/or firing duration, sets and/or subsets of the plurality of energy guides can also be powered at any given point in time. Each set or subset of the plurality of energy guides can include one or more of the plurality of energy guides. Thus, at any given point in time, power can be directed to one or more of the plurality of energy guides to alternatively create a first firing sequence, a second firing sequence, a third firing sequence, a fourth firing sequence, etc. Moreover, in various applications of the present invention, each firing sequence of the energy guides in such sets and subsets of the plurality of energy guides can be different than one or more of the other firing sequences of the energy guides.

The system controller 126 can further be configured to control operation of other components of the catheter system 100 such as the positioning of the catheter 102 and/or the guide distal end 122D of the energy guides 122A adjacent to the treatment site 106, the inflation of the balloon 104 with the catheter fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 129.

The GUI 127 is accessible by the user or operator of the catheter system 100. The GUI 127 is electrically connected to the system controller 126. With such design, the GUI 127 can be used by the user or operator to ensure that the catheter system 100 is effectively utilized to impart pressure onto and induce fractures at the treatment site(s) 106. The GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications and/or desires of the user or operator.

Where applicable, the multiplexer 128 can be configured to selectively and/or alternatively direct light energy from the energy source 124 to each of the energy guides 122A in the energy guide bundle 122. More particularly, the multiplexer 128 is configured to receive light energy from the energy source 124, such as a single source beam 124A from a single laser source, and selectively and/or alternatively direct such light energy in the form of individual guide beams 124B to each of the energy guides 122A in the energy guide bundle 122. As such, the multiplexer 128 enables a single energy source 124 to be channeled separately in any desired firing sequence or pattern through a plurality of energy guides 122A such that the catheter system 100 is able to impart pressure onto and induce fractures in vascular lesions 106A at the treatment site 106 within or adjacent to a vessel wall 108A of the blood vessel 108 in a desired manner. More particularly, as described in detail herein, the desired firing sequence for the multiplexer 128 directing individual guide beams 124B to any plurality of energy guides 122A can be tailored to generate a desired advancing wavefront 835 that can be utilized to target the vascular lesions 106A at the treatment site 106 in a desired manner.

In certain embodiments, as shown, the catheter system 100 can include one or more optical elements 147 for purposes of directing the light energy, such as the source beam 124A, from the energy source 124 to the multiplexer 128.

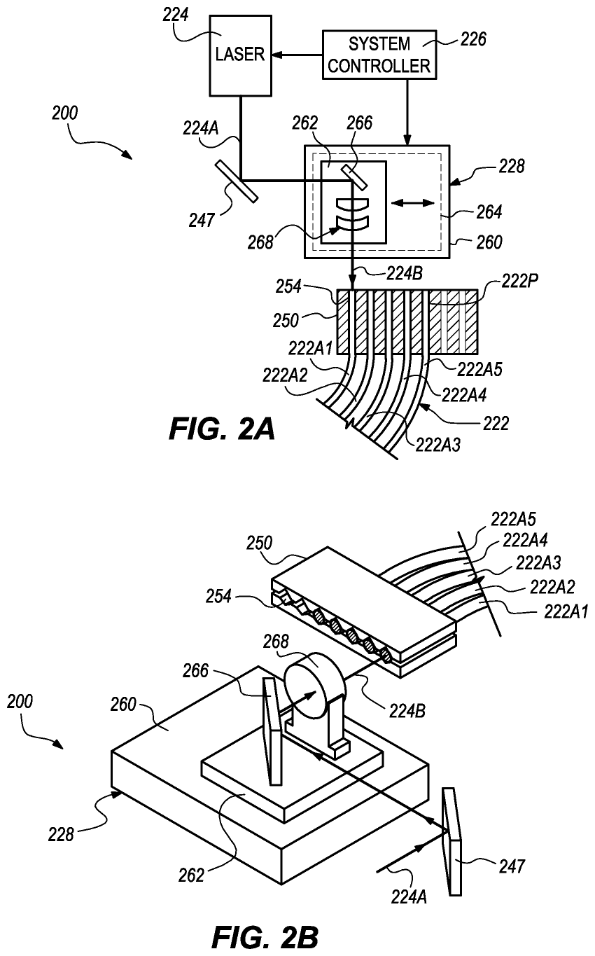

As described herein, the multiplexer 128 can have any suitable design for purposes of selectively and/or alternatively directing the light energy from the energy source 124 to each of the energy guides 122A of the energy guide bundle 122. More particularly, in many implementations, the multiplexer 128 can have any suitable design for purposes of selectively and/or alternatively directing the light energy from the energy source 124 to each of the energy guides 122A of the energy guide bundle 122 in a desired firing sequence for purposes of generating the desired advancing wavefront 835. Various non-exclusive alternative embodiments of the multiplexer 128 are described in detail herein below in relation to FIGS. 2A-7.

As shown in FIG. 1, the handle assembly 129 can be positioned at or near the proximal portion 114 of the catheter system 100, and/or near the source manifold 136. In this embodiment, the handle assembly 129 is coupled to the balloon 104 and is positioned spaced apart from the balloon 104. Alternatively, the handle assembly 129 can be positioned at another suitable location.

The handle assembly 129 is handled and used by the user or operator to operate, position and control the catheter 102. The design and specific features of the handle assembly 129 can vary to suit the design requirements of the catheter system 100. In the embodiment illustrated in FIG. 1, the handle assembly 129 is separate from, but in electrical and/or fluid communication with one or more of the system controller 126, the light source 124, the fluid pump 138, the GUI 127, and the multiplexer 128. In some embodiments, the handle assembly 129 can integrate and/or include at least a portion of the system controller 126 within an interior of the handle assembly 129. For example, as shown, in certain such embodiments, the handle assembly 129 can include circuitry 155 that can form at least a portion of the system controller 126. In one embodiment, the circuitry 155 can include a printed circuit board having one or more integrated circuits, or any other suitable circuitry. In an alternative embodiment, the circuitry 155 can be omitted, or can be included within the system controller 126, which in various embodiments can be positioned outside of the handle assembly 129, such as within the system console 123. It is understood that the handle assembly 129 can include fewer or additional components than those specifically illustrated and described herein.

FIG. 2A is a simplified schematic top view illustration of a portion of an embodiment of the catheter system 200. More particularly, FIG. 2A illustrates a plurality of energy guides, such as a first energy guide 222A1, a second energy guide 222A2, a third energy guide 222A3, a fourth energy guide 222A4 and a fifth energy guide 222A5, an energy source 224, a system controller 226, and an embodiment of the multiplexer 228. The energy guides 222A1-222A5, the energy source 224 and the system controller 226 are substantially similar in design and function as previously described. Accordingly, such components will not be described in detail in relation to the embodiment illustrated in FIG. 2A. It is further appreciated that certain components of the system console 123 illustrated and described above in relation to FIG. 1, such as the power source 125 and the GUI 127, are not illustrated in FIG. 2A for purposes of simplicity and ease of illustration, but would typically be included in many embodiments.

The multiplexer 228 is configured to receive light energy in the form of the source beam 224A, such as a pulsed source beam, from the energy source 224, and selectively and/or alternatively direct the light energy in the form of individual guide beams 224B in any desired firing sequence and/or pattern (whether predetermined or otherwise) to each of the energy guides 222A1-222A5 under control of the system controller 226. As such, as shown in FIG. 2A, the multiplexer 228 is operatively and/or optically coupled in optical communication to the energy guide bundle 222 and/or to the plurality of energy guides 222A1-222A5.

As illustrated, a guide proximal end 222P of each of the plurality of energy guides 222A1-222A5 is retained within a guide coupling housing 250, such as within guide coupling slots 254 that are formed into the guide coupling housing 250. In various embodiments, the guide coupling housing 250 is configured to be selectively coupled to the system console 123 (illustrated in FIG. 1) so that the guide coupling slots 254, and thus the energy guides 222A1-222A5, are maintained in a desired fixed position relative to the multiplexer 228 during use of the catheter system 200. In some embodiments, the guide coupling slots 254 are provided in the form of V-grooves, such as in a V-groove ferrule block commonly used in multichannel fiber optics communication systems. Alternatively, the guide coupling slots 254 can have another suitable design.

It is appreciated that the guide coupling housing 250 can have any suitable number of guide coupling slots 254, which can be positioned and/or oriented relative to one another in any suitable manner, so as to better align the guide coupling slots 254 and thus the energy guides 222A1-222A5 relative to the multiplexer 228. In the embodiment illustrated in FIG. 2A, the guide coupling housing 250 includes seven guide coupling slots 254 that are spaced apart in a linear arrangement relative to one another, with precise interval spacing between adjacent guide coupling slots 254. Thus, in such embodiment, the guide coupling housing 250 is capable of retaining the guide proximal end 222P of up to seven energy guides (although only five energy guides 222A1-222A5 are specifically shown in FIG. 2A). Alternatively, the guide coupling housing 250 can have a different number of guide coupling slots, such as greater than seven or fewer than seven, and/or the guide coupling slots 254 can be arranged in a different manner relative to one another.

The design of the multiplexer 228 can be varied depending on the requirements of the catheter system 200, the relative positioning of the energy guides 222A1-222A5, and/or to suit the desires of the user or operator of the catheter system 200. In the embodiment illustrated in FIG. 2A, the multiplexer 228 includes one or more of a multiplexer base 260, a multiplexer stage 262, a stage mover 264 (illustrated in phantom), a redirector 266, and coupling optics 268. Alternatively, the multiplexer 228 can include more components or fewer components than those specifically illustrated in FIG. 2A.

During use of the catheter system 200, the multiplexer base 260 is fixed in position relative to the energy source 224 and the energy guides 222A1-222A5. In this embodiment, the multiplexer stage 262 is movably supported on the multiplexer base 260. More particularly, the stage mover 264 is configured to move the multiplexer stage 262 relative to the multiplexer base 260. As shown in FIG. 2A, the redirector 266 and the coupling optics 268 are mounted on and/or retained by the multiplexer stage 262. Thus, movement of the multiplexer stage 262 relative to the multiplexer base 260 results in corresponding movement of the redirector 266 and the coupling optics 268 relative to the fixed multiplexer base 260. In some embodiments, with the energy guides 222A1-222A5 being fixed in position relative to the multiplexer base 260, movement of the multiplexer stage 262 results in corresponding movement of the redirector 266 and the coupling optics 268 relative to the energy guides 222A1-222A5.

In various embodiments, the multiplexer 228 is configured to precisely align the coupling optics 268 with each of the energy guides 222A1-222A5 such that the source beam 224A generated by the energy source 224 can be precisely directed and focused by the multiplexer 228 as a corresponding guide beam 224B to each of the energy guides 222A1-222A5. In its simplest form, as shown in FIG. 2A, the multiplexer 228 uses a precision mechanism, such as the stage mover 264, to translate the coupling optics 268 along a linear path. This approach requires a single degree of freedom. In certain embodiments, the linear translation mechanism, such as the stage mover 264, and/or the multiplexer stage 262 can be equipped with mechanical stops so that the coupling optics 268 can be precisely aligned with the position of each of the energy guides 222A1-222A5 in any desired firing sequence and/or pattern (whether predetermined or otherwise). Alternatively, the stage mover 264 can be electronically controlled to line the beam path of the guide beam 224B in any desired firing sequence and/or pattern with each individual energy guide 222A1-222A5 that is retained, in part, within the guide coupling housing 250.

The multiplexer stage 262 is configured to carry the necessary optics, such as the redirector 266 and the coupling optics 268, to direct and focus the light energy generated by the energy source 224 onto each energy guide 222A1-222A5 for optimal coupling. With such design, the low divergence of the guide beam 224A over the short distance of motion of the translated multiplexer stage 262 has minimum impact on coupling efficiency to the energy guide 222A1-222A5.

During operation, the stage mover 264 drives the multiplexer stage 262 to align the beam path of the guide beam 224B with a selected energy guide 222A1-222A5 and then the system controller 226 fires the energy source 224 in pulsed or semi-CW mode. The stage mover 264 then steps the multiplexer stage 262 to the next stop, such as to the next desired energy guide 222A1-222A5, and the system controller 226 again fires the energy source 224. This process is repeated as desired so that light energy in the form of the guide beams 224B is directed onto each of the energy guides 222A1-222A5 in a desired firing sequence and/or pattern. It is appreciated that the stage mover 264 can move the multiplexer stage 262 so that it is aligned with any of the energy guides 222A1-222A5, then the system controller 226 fires the energy source 224. In this manner, the multiplexer 228 can achieve a firing sequence through the energy guides 222A1-222A5 in any desired firing pattern. It is further appreciated that the system controller 226 controlling the multiplexer 228 to achieve the desired firing sequence plays an important role in the generation of the desired advancing wavefront 835 (illustrated in FIG. 8A).

In this embodiment, the stage mover 264 can have any suitable design for purposes of moving the multiplexer stage 262 in a linear manner relative to the multiplexer base 260. More particularly, the stage mover 264 can be any suitable type of linear translation mechanism.

As shown in FIG. 2A, the catheter system 200 can further include an optical element 247, such as a reflecting or redirecting element like a mirror, that reflects the source beam 224A from the energy source 224 so that the source beam 224A is directed toward the multiplexer 228. In one embodiment, as shown, the optical element 247 can be positioned along the beam path to redirect the source beam 224A by approximately 90 degrees so that the source beam 224A is directed toward the multiplexer 228. Alternatively, the optical element 247 can redirect the source beam 224A by more than 90 degrees or less than 90 degrees. Still alternatively, the catheter system 200 can be designed without the optical element 247, and the energy source 224 can direct the source beam 224A directly toward the multiplexer 228.

As illustrated in this embodiment, the source beam 224A being directed toward the multiplexer 228 initially impinges on the redirector 266, which is configured to redirect the source beam 224A toward the coupling optics 268. In some embodiments, the redirector 266 redirects the source beam 224A by approximately 90 degrees toward the coupling optics 268. Alternatively, the redirector 266 can redirect the source beam 224A by more than 90 degrees or less than 90 degrees toward the coupling optics 268. Thus, the redirector 266 that is mounted on the multiplexer stage 262 is configured to direct the source beam 224A through the coupling optics 268 so that individual guide beams 224B are focused into the individual energy guides 222A1-222A5 in the guide coupling housing 250.

The coupling optics 268 can have any suitable design for purposes of focusing the individual guide beams 224B onto each of the energy guides 222A1-222A5. In one embodiment, the coupling optics 268 includes two lenses that are specifically configured to focus the individual guide beams 224B as desired. Alternatively, the coupling optics 268 can have another suitable design.

In certain non-exclusive alternative embodiments, the steering of the source beam 224A so that it is properly directed and focused onto each of the energy guides 222A1-222A5 can be accomplished using mirrors that are attached to optomechanical scanners, X-Y galvanometers or other multi-axis beam steering devices.

Still alternatively, although FIG. 2A illustrates that the energy guides 222A1-222A5 are fixed in position relative to the multiplexer base 260, in some embodiments, it is appreciated that the energy guides 222A1-222A5 can be configured to move relative to coupling optics 268 that are fixed in position. In such embodiments, the guide coupling housing 250 itself would move. In one non-exclusive example, the guide coupling housing 250 can be carried by a linear translation stage, and the system controller 226 can control the linear translation stage to move in a stepped manner so that the energy guides 222A1-222A5 are each aligned, in a desired firing sequence or pattern, with the coupling optics and the guide beams 224B. While such an embodiment can be effective, it is further appreciated that additional protection and controls would be required to make it safe and reliable as the guide coupling housing 250 moves relative to the coupling optics 268 of the multiplexer 228 during use.

FIG. 2B is a simplified schematic perspective view illustration of a portion of the catheter system 200 and the multiplexer 228 illustrated in FIG. 2A. In particular, FIG. 2B illustrates another view of the guide coupling housing 250, with the guide coupling slots 254, that is configured to retain a portion of each of the energy guides 222A1-222A5; the optical element 247 that initially redirects the source beam 224A from the energy source 224 (illustrated in FIG. 2A) toward the multiplexer 228; and the multiplexer 228, including the multiplexer base 260, the multiplexer stage 262, the redirector 266 and the coupling optics 268, that receives the source beam 224A and then directs and focuses individual guide beams 224B in any desired firing sequence and/or pattern toward each of the energy guides 222A-222E. It is appreciated that the stage mover 264 is not illustrated in FIG. 2B for purposes of simplicity and ease of illustration.

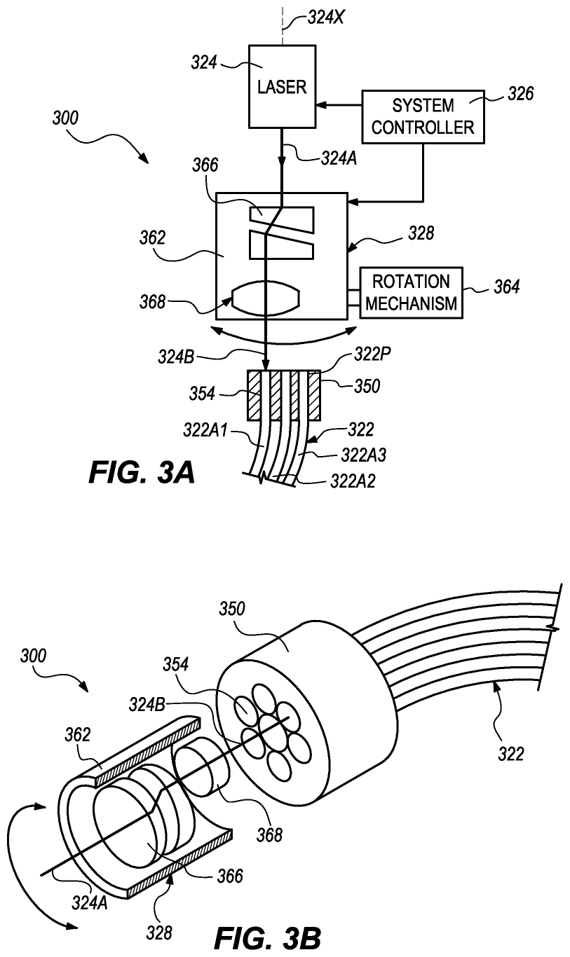

FIG. 3A is a simplified schematic top view illustration of a portion of an embodiment of the catheter system 300 including another embodiment of the multiplexer 328. More particularly, FIG. 3A illustrates a plurality of energy guides, such as a first energy guide 322A1, a second energy guide 322A2 and a third energy guide 322A3, an energy source 324, a system controller 326, and the multiplexer 328 that receives light energy in the form of a source beam 324A from the energy source 324 and selectively and/or alternatively directs the light energy in the form of individual guide beams 324B in any desired firing sequence and/or pattern to each of the energy guides 322A1-322A3, under control of the system controller 326. The energy guides 322A1-322A3, the energy source 324 and the system controller 326 are substantially similar in design and function as described in detail herein above. Accordingly, such components will not be described in detail in relation to the embodiment illustrated in FIG. 3A. It is further appreciated that certain components of the system console 123 illustrated and described above in relation to FIG. 1, such as the power source 125 and the GUI 127, are not illustrated in FIG. 3A for purposes of simplicity and ease of illustration, but would typically be included in many embodiments.

As with previous embodiments, the multiplexer 328 is configured to receive light energy in the form of the source beam 324A, such as a single pulsed source beam, from the energy source 324 and selectively and/or alternatively direct the light energy in the form of individual guide beams 324B in any desired firing sequence and/or pattern to each of the energy guides 322A1-322A3 under control of the system controller 326. As such, as shown in FIG. 3A, the multiplexer 328 is operatively and/or optically coupled in optical communication to the energy guide bundle 322 and/or to the plurality of energy guides 322A1-322A3.

As illustrated, a guide proximal end 322P of each of the plurality of energy guides 322A1-322A3 is retained within a guide coupling housing 350, such as within guide coupling slots 354 that are formed into the guide coupling housing 350. In various embodiments, the guide coupling housing 350 is configured to be selectively coupled to the system console 123 (illustrated in FIG. 1) so that the guide coupling slots 354, and thus the energy guides 322A1-322A3, are maintained in a desired fixed position relative to the multiplexer 328 during use of the catheter system 300.

Referring now to FIG. 3B, FIG. 3B is a simplified schematic perspective view illustration of a portion of the catheter system 300 and the multiplexer 328 illustrated in FIG. 3A. As shown in FIG. 3B, the guide coupling housing 350 can be substantially cylindrical-shaped. It is appreciated that the guide coupling housing 350 can have any suitable number of guide coupling slots 354, which can be positioned and/or oriented relative to one another in any suitable manner, so as to best align the guide coupling slots 354 and thus the energy guides 322A1-322A3 of the energy guide bundle 322 relative to the multiplexer 328. In the embodiment illustrated in FIG. 3B, the guide coupling housing 350 includes seven guide coupling slots 354 that are arranged in a circular and/or hexagonal packed pattern. Thus, in such embodiment, the guide coupling housing 350 is capable of retaining the guide proximal end of up to seven energy guides. Alternatively, the guide coupling housing 350 can have a different number of guide coupling slots, such as greater than seven or less than seven, and/or the guide coupling slots 354 can be arranged in a different manner relative to one another, such as in another suitable circular periodic pattern.

Returning to FIG. 3A, in this embodiment, the multiplexer 328 includes one or more of a multiplexer stage 362, a stage mover 364, a redirector 366, and coupling optics 368. Alternatively, the multiplexer 328 can include more components or fewer components than those specifically illustrated in FIG. 3A.

As shown in the embodiment illustrated in FIG. 3A, the stage mover 364 is configured to move the multiplexer stage 362 in a rotational manner. More particularly, in this embodiment, the multiplexer stage 362 and/or the stage mover 364 requires a single rotational degree of freedom. As illustrated, the multiplexer stage 362 and the guide coupling housing 350 are aligned on a central axis 324X of the energy source 324. As such, the multiplexer stage 362 is configured to be rotated by the stage mover 364 about the central axis 324X.

The redirector 366 and the coupling optics 368 are mounted on and/or retained by the multiplexer stage 362. During use of the catheter system 300, the source beam 324A is initially directed toward the multiplexer 328 and/or the multiplexer stage 362, along the central axis 324X of the energy source 324. Subsequently, the redirector 366 is configured to deviate the source beam 324A a fixed distance laterally off the central axis 324X of the energy source 324, such that the source beam 324A is directed in a direction that is substantially parallel to and spaced apart from the central axis 324X. More specifically, the redirector 366 deviates the source beam 324A to coincide with the radius of the circular pattern of the energy guides 322A1-322A3 in the guide coupling housing 350. As the multiplexer stage 362 is rotated, the source beam 324A that is directed through the redirector 366 traces out a circular path.

It is appreciated that the redirector 366 can have any suitable design. For example, in certain non-exclusive alternative embodiments, the redirector 366 can be provided in the form of an anamorphic prism pair, a pair of wedge prisms, or a pair of close-spaced right angle mirrors or prisms. Alternatively, the redirector 366 can include another suitable configuration of optics in order to achieve the desired lateral beam offset.

The coupling optics 368 are also mounted on and/or retained by the multiplexer stage 362. As with the previous embodiments, the coupling optics 368 are configured to focus the individual guide beams 324B onto each of the energy guides 322A1-322A3 in the energy guide bundle 322 retained, in part, within the guide coupling housing 350 for optimal coupling.

The multiplexer 328 is configured to precisely align the coupling optics 368 with each of the energy guides 322A1-322A3 such that the source beam 324A generated by the energy source 324 can be precisely directed and focused by the multiplexer 328 as a corresponding guide beam 324B to each of the energy guides 322A1-322A3. In certain embodiments, the stage mover 364 and/or the multiplexer stage 362 can be equipped with mechanical stops so that the coupling optics 368 can be precisely aligned with the position of each of the energy guides 322A1-322A3 in any desired firing sequence and/or pattern. Alternatively, the stage mover 364 can be electronically controlled, such as by using stepper motors or a piezo-actuated rotational stage, to line the beam path of the guide beam 324B in any desired firing sequence and/or pattern with each individual energy guide 322A1-322A3 that is retained, in part, within the guide coupling housing 350.

During use of the catheter system 300, the stage mover 364 drives the multiplexer stage 362 to couple the guide beam 324B with a selected energy guide 322A1-322A3 and then the system controller 326 fires the energy source 324 in pulsed or semi-CW mode. The stage mover 364 then steps the multiplexer stage 362 angularly to the next stop, such as to the next desired energy guide 322A1-322A3, and the system controller 326 again fires the energy source 324. This process is repeated as desired so that light energy in the form of the guide beams 324B is directed onto each of the energy guides 322A1-322A3 in a desired firing sequence and/or pattern. It is appreciated that the stage mover 364 can move the multiplexer stage 362 so that it is aligned with any of the energy guides 322A1-322A3, then the system controller 326 fires the energy source 324. In this manner, the multiplexer 328 can achieve a particular firing sequence through the energy guides 322A1-322A3 or fire in any desired firing sequence or pattern relative to the energy guides 322A1-322A3. It is further appreciated that the system controller 326 controlling the multiplexer 328 to achieve the desired firing sequence plays an important role in the generation of the desired advancing wavefront 835 (illustrated in FIG. 8A).

In this embodiment, the stage mover 364 can have any suitable design for purposes of moving the multiplexer stage 362 in a rotational manner about the central axis 324X. More particularly, the stage mover 364 can be any suitable type of rotational mechanism.

Alternatively, although FIG. 3A illustrates that the energy guides 322A1-322A3 are fixed in position relative to the multiplexer stage 362, in some embodiments, it is appreciated that the energy guides 322A1-322A3 can be configured to move and/or rotate relative to coupling optics 368 that are fixed in position. In such embodiments, the guide coupling housing 350 itself would move, such that the guide coupling housing 350 can be rotated about the central axis 324X, and the system controller 326 can control the rotational stage to move in a stepped manner so that the energy guides 322A1-322A3 are each aligned, in a desired firing sequence and/or pattern, with the coupling optics and the guide beams 324B. In such embodiment, the guide coupling housing 350 would not be continuously rotated, but would be rotated a fixed number of degrees and then counter-rotated to avoid the winding of the energy guides 322A1-322A3.

Returning again to FIG. 3B, another view of the guide coupling housing 350 is shown. FIG. 3B illustrates the guide coupling slots 354, that are configured to retain a portion of each of the energy guides, and the multiplexer 328, including the multiplexer stage 362, the redirector 366 and the coupling optics 368, that receives the source beam 324A and then directs and focuses individual guide beams 324B in any desired firing sequence and/or firing pattern toward each of the energy guides 322A1-322A3. It is appreciated that the stage mover 364 is not illustrated in FIG. 3B for purposes of simplicity and ease of illustration.

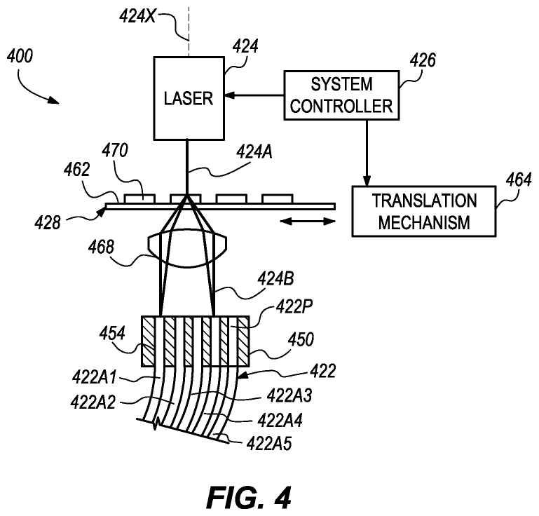

FIG. 4 is a simplified schematic top view illustration of a portion of the catheter system 400 and still another embodiment of the multiplexer 428. More particularly, FIG. 4 illustrates a plurality of energy guides, such as a first energy guide 422A1, a second energy guide 422A2, a third energy guide 422A3, a fourth energy guide 422A4 and a fifth energy guide 422A5, an energy source 424, a system controller 426, and the multiplexer 428 that receives light energy in the form of a source beam 424A from the energy source 424 and selectively and/or alternatively directs the light energy in the form of individual guide beams 424B in any desired firing sequence and/or pattern to each of the energy guides 422A1-422A5, under control of the system controller 426. The energy guides 422A1-422A5, the energy source 424 and the system controller 426 are substantially similar in design and function as described in detail herein above. Accordingly, such components will not be described in detail in relation to the embodiment illustrated in FIG. 4. It is further appreciated that certain components of the system console 123 illustrated and described above in relation to FIG. 1, such as the power source 125 and the GUI 127, are not illustrated in FIG. 4 for purposes of simplicity and ease of illustration, but would typically be included in many embodiments.

The multiplexer 428 is again configured to receive light energy in the form of the source beam 424A, such as a single pulsed source beam, from the energy source 424 and selectively and/or alternatively direct the light energy in the form of individual guide beams 424B in any desired firing sequence and/or pattern to each of the energy guides 422A1-422A5, under control of the system controller 426. As such, as shown in FIG. 4, the multiplexer 428 is operatively and/or optically coupled in optical communication to the energy guide bundle 422 and/or to the plurality of energy guides 422A1-422A5.

As illustrated, a guide proximal end 422P of each of the plurality of energy guides 422A1-422A5 is retained within a guide coupling housing 450, such as within guide coupling slots 454 that are formed into the guide coupling housing 450. In various embodiments, the guide coupling housing 450 is configured to be selectively coupled to the system console 123 (illustrated in FIG. 1) so that the guide coupling slots 454, and thus the energy guides 422A1-422A5, are maintained in a desired fixed position relative to the multiplexer 428 during use of the catheter system 400. It is appreciated that the guide coupling housing 450 can have any suitable number of guide coupling slots 454. In the embodiment illustrated in FIG. 4, five guide coupling slots 454 are visible within the guide coupling housing 450. Thus, in such embodiment, the guide coupling housing 450 is capable of retaining the guide proximal end 422P of up to five energy guides. Alternatively, the guide coupling housing 450 can have a different number of guide coupling slots 454, such as greater than five or less than five guide coupling slots 454.

In the embodiment illustrated in FIG. 4, the multiplexer 428 includes one or more of a multiplexer stage 462, a stage mover 464, one or more diffractive optical elements 470 (or “DOE”), and coupling optics 468. Alternatively, the multiplexer 428 can include more components or fewer components than those specifically illustrated in FIG. 4.

As shown, the diffractive optical elements 470 are mounted on and/or retained by the multiplexer stage 462. The stage mover 464 is configured to move the multiplexer stage 462, such as translationally, such that each of the one or more diffractive optical elements 470 are selectively and/or alternatively positioned in the beam path of the source beam 424A from the energy source 424.

During use of the catheter system 400, each of the one or more diffractive optical elements 470 is configured to separate the source beam 424A into one, two, three or more individual guide beams 424B. It is appreciated that the diffractive optical elements 470 can have any suitable design. For example, in certain non-exclusive embodiments, the diffractive optical elements 470 can be created using arrays of micro-prisms, micro-lenses, or other patterned diffractive elements.

It is appreciated that there are many possible physical configurations, patterns, or setups to organize the energy guides 422A1-422A5 in the guide coupling housing 450 using this approach. One such configuration for the energy guides 422A1-422A5 within the guide coupling housing 450 would be a hexagonal, closely-packed configuration, somewhat similar to that illustrated in FIGS. 3A and 3B. Alternatively, the energy guides 422A1-422A5 within the guide coupling housing 450 could also be arranged in a square, rectangular, triangular, pentagonal, linear, circular, or any other suitable geometric or non-geometric configuration.

As shown in FIG. 4, the guide coupling housing 450 can be aligned on the central axis 424X of the energy source 424, with the diffractive optical elements 470 mounted on the multiplexer stage 462 being inserted along the beam path between the energy source 424 and the guide coupling housing 450. The coupling optics 468 are also positioned along the central axis 424X of the energy source 424, and the coupling optics are positioned between the diffractive optical elements 470 and the guide coupling housing 450.

During operation, the source beam 424A impinging on one of the plurality of diffractive optical elements 470 splits the source beam 424A into two or more deviated beams, such as two or more guide beams 424B. These guide beams 424B are, in turn, directed and focused by the coupling optics 468 down onto the individual energy guides 422A1-422A5 that are retained in the guide coupling housing 450. In one configuration, the diffractive optical element 470 would split the source beam 424A into as many energy guides as are present within the single-use device. In such configuration, the power in each guide beam 424B is based on the number of guide beams 424B that are generated from the single source beam 424A minus scattering and absorption losses. Alternatively, the diffractive optical element 470 can be configured to split the source beam 424A so that guide beams 424B are directed into any single energy guide or any selected multiple energy guides. Thus, the multiplexer stage 462 can be configured to retain a plurality of diffractive optical elements 470, such as with multiple diffractive optical element patterns etched on a single plate, to provide options for the user or operator for coupling the guide beams 424B to the desired number and pattern of energy guides. In such embodiments, the desired firing sequence can be achieved by moving the multiplexer stage 462 with the stage mover 464, such as translationally, so that the desired diffractive optical element 470 is positioned in the beam path of the source beam 424A between the energy source 424 and the coupling optics 468.

It is further appreciated that the system controller 426 controlling the multiplexer 428 to achieve the desired firing sequence plays an important role in the generation of the desired advancing wavefront 835 (illustrated in FIG. 8A).

As with the previous embodiments, the coupling optics 468 can have any suitable design for purposes of focusing the individual guide beams 424B, or multiple guide beams 424B simultaneously, onto the desired energy guides 422A1-422A5.

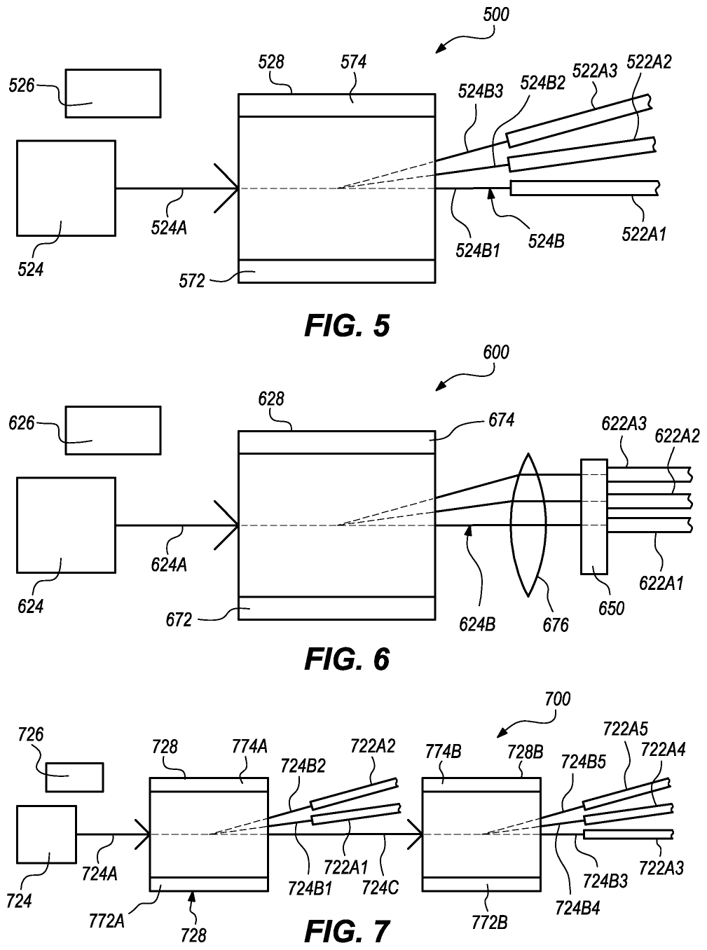

FIG. 5 is a simplified schematic top view illustration of a portion of the catheter system 500 and yet another embodiment of the multiplexer 528. More particularly, FIG. 5 illustrates a plurality of energy guides, such as a first energy guide 522A1, a second energy guide 522A2 and a third energy guide 522A3, an energy source 524, a system controller 526, and the multiplexer 528 that receives light energy in the form of a source beam 524A from the energy source 524 and selectively and/or alternatively directs the light energy in the form of individual guide beams 524B in any desired firing sequence and/or pattern to each of the energy guides 522A1-522A3, under control of the system controller 526. The energy guides 522A1-522A3, the energy source 524 and the system controller 526 are substantially similar in design and function as described in detail herein above. Accordingly, such components will not be described in detail in relation to the embodiment illustrated in FIG. 5. It is further appreciated that certain components of the system console 123 illustrated and described above in relation to FIG. 1, such as the power source 125 and the GUI 127, are not illustrated in FIG. 5 for purposes of simplicity and ease of illustration, but would typically be included in many embodiments.

The multiplexer 528 is again configured to receive light energy in the form of the source beam 524A, such as a single pulsed source beam, from the energy source 524 and selectively and/or alternatively direct the light energy in the form of individual guide beams 524B in any desired firing sequence and/or pattern to each of the energy guides 522A1-522A3, under control of the system controller 526. As such, as shown in FIG. 5, the multiplexer 528 is operatively and/or optically coupled in optical communication to the plurality of energy guides 522A1-522A3.

However, as illustrated in FIG. 5, the multiplexer 528 has a different design than any of the previous embodiments. In some embodiments, it may be desirable to design the multiplexer 528 to receive the source beam 524A from a single energy source 524 and selectively and/or alternatively direct the light energy in the form of individual guide beams 524B in any desired firing sequence and/or pattern to each of the energy guides 522A1-522A3 in a manner that is easily reconfigurable and that does not involve moving parts. For example, using an acousto-optic deflector (AOD) as the multiplexer 528 can allow the entire output of a single energy source 524, such as a single laser, to be directed into a plurality of individual energy guides 522A1-522A3. The guide beam 524B can be re-targeted to a different energy guide 522A1-522A3 within microseconds by simply changing the driving frequency input into the multiplexer 528 (the AOD), and with a pulsed laser such as a Nd:YAG, this switching can easily occur between pulses. In such embodiments, the deflection angle (O) of the multiplexer 528 can be defined as follows:
Deflection angle (⊖)=∧f/v where
∧=Optical Wavelength
f=acoustic drive frequency
v=speed of sound in modulator

As shown in FIG. 5, the source beam 524A is directed from the energy source 524 toward the multiplexer 528, and is subsequently redirected due to the generated deflection angle as a desired guide beam 524B to each of the energy guides 522A1-522A3. More specifically, as illustrated, when the multiplexer 528 generates a first deflection angle for the source beam 524A, a first guide beam 524B1 is directed to the first energy guide 522A1; when the multiplexer 528 generates a second deflection angle for the source beam 524A, a second guide beam 524B2 is directed to the second energy guide 522A2; and when the multiplexer 528 generates a third deflection angle for the source beam 524A, a third guide beam 524B3 is directed to the third energy guide 522A3. It is appreciated that, as illustrated, any desired deflection angle can effectively include no deflection angle at all, such that the guide beam 524B can be directed to continue along the same axial beam path as the source beam 524A.

In this embodiment, the multiplexer 528 (AOD) includes a transducer 572 and an absorber 574 that cooperate to generate the desired driving frequency that can, in turn, generate the desired deflection angle so that the source beam 524A is redirected as the desired guide beam 524B toward the desired energy guide 522A1-522A3. More particularly, the multiplexer 528 is configured to spatially control the source beam 524A. In the operation of the multiplexer 528, the power driving the acoustic transducer 572 is kept on, at a constant level, while the acoustic frequency is varied to deflect the source beam 524A to different angular positions that define the guide beams 524B1-524B3. Thus, the multiplexer 528 makes use of the acoustic frequency-dependent diffraction angle, such as described above.

It is further appreciated that the system controller 526 controlling the multiplexer 528 to achieve the desired firing sequence plays an important role in the generation of the desired advancing wavefront 835 (illustrated in FIG. 8A).

FIG. 6 is a simplified schematic top view illustration of a portion of the catheter system 600 and still another embodiment of the multiplexer 628. More particularly, FIG. 6 illustrates a plurality of energy guides, such as a first energy guide 622A1, a second energy guide 622A2 and a third energy guide 622A3, an energy source 624, a system controller 626, and the multiplexer 628 that receives light energy in the form of a source beam 624A, such as a single pulsed source beam, from the energy source 624 and selectively and/or alternatively directs the light energy in the form of individual guide beams 624B in any desired firing sequence and/or pattern to each of the energy guides 622A1-622A3, under control of the system controller 626. The energy guides 622A1-622A3, the energy source 624 and the system controller 626 are substantially similar in design and function as described in detail herein above. Accordingly, such components will not be described in detail in relation to the embodiment illustrated in FIG. 6. It is further appreciated that certain components of the system console 123 illustrated and described above in relation to FIG. 1, such as the power source 125 and the GUI 127, are not illustrated in FIG. 6 for purposes of simplicity and ease of illustration, but would typically be included in many embodiments.

It is appreciated that the multiplexer 628 illustrated in FIG. 6 is substantially similar to the multiplexer 528 illustrated and described in relation to FIG. 5. For example, as shown in FIG. 6, the multiplexer 628 again includes a transducer 672 and an absorber 674 that cooperate to generate the desired driving frequency that can, in turn, generate the desired deflection angle so that the source beam 624A is redirected as the desired guide beam 624B toward the desired energy guide 622A1-622A3. However, in this embodiment, the multiplexer 628 further includes an optical element 676 that is usable to transform the angular separation between the guide beams 624B into a linear offset.

In some embodiments, in order to improve the angular resolution and the efficiency of the catheter system 600, the input laser 624 should be collimated with a diameter close to filling the aperture of the multiplexer 628 (the AOD). The smaller the divergence of the input, the greater number of discrete outputs can be generated. The angular resolution of such a device is quite good, but the total angular deflection is limited. To allow a sufficient number of energy guides 622A1-622A3 of finite size to be accessed by a single energy source 624 and a single source beam 624A, there are a number of means to improve the separation of the different output. For example, as shown in FIG. 6, after the individual guide beams 624B separate, the optical element 676, such as a lens, can be used to transform the angular separation between the guide beams 624B into a linear offset, and can be used to direct the guide beams 624B into closely spaced energy guides 622A1-622A3, such as when the energy guides 622A1-622A3 are held in close proximity to one another within a guide coupling housing 650. In certain embodiments, folding mirrors can be used to allow adequate propagation distance to separate the different beam paths of the guide beams 624B within a limited volume.

As with the previous embodiments, it is appreciated that the system controller 626 controlling the multiplexer 628 to achieve the desired firing sequence plays an important role in the generation of the desired advancing wavefront 835 (illustrated in FIG. 8A).

FIG. 7 is a simplified schematic top view illustration of a portion of the catheter system 700 and still yet another embodiment of the multiplexer 728. More particularly, FIG. 7 illustrates a plurality of energy guides, such as a first energy guide 722A1, a second energy guide 722A2, a third energy guide 722A3, a fourth energy guide 722A4 and a fifth energy guide 722A5, an energy source 724, a system controller 726, and the multiplexer 728 that receives light energy in the form of a source beam 724A, such as a single pulsed source beam, from the energy source 724 and selectively and/or alternatively directs the light energy in the form of individual guide beams 724B in any desired firing sequence and/or pattern to each of the energy guides 722A1-722A5, under control of the system controller 726. The energy guides 722A1-722A5, the energy source 724 and the system controller 726 are substantially similar in design and function as described in detail herein above. Accordingly, such components will not be described in detail in relation to the embodiment illustrated in FIG. 7. It is further appreciated that certain components of the system console 123 illustrated and described above in relation to FIG. 1, such as the power source 125 and the GUI 127, are not illustrated in FIG. 7 for purposes of simplicity and ease of illustration, but would typically be included in many embodiments.

It is appreciated that the manner for multiplexing the source beam 724A into multiple guide beams 724B illustrated in FIG. 7 is somewhat similar to how the source beam 524 was multiplexed into multiple guide beams 524B as illustrated and described in relation to FIG. 5. However, in this embodiment, the multiplexer 728 includes a pair of acousto-optic deflectors (AODs), such as a first acousto-optic deflector 728A and a second acousto-optic deflector 728B, that are positioned in series with one another. With such design, the multiplexer 728 may be able to access additional energy guides. It is further appreciated that the multiplexer 728 can include more than two acousto-optic deflectors, if desired, to be able to access even more energy guides.

In the embodiment shown in FIG. 7, the source beam 724A is initially directed toward the first AOD 728A. The first AOD 728A is utilized to deflect the source beam 724A to generate a first guide beam 724B1 that is directed toward the first energy guide 722A1, and a second guide beam 724B2 that is directed toward the second energy guide 722A2. The first AOD 728A also allows an undeviated beam to be transmitted through the first AOD 728A as a transmitted beam 724C that is directed toward the second AOD 728B. Subsequently, the second AOD 728B is utilized to deflect the transmitted beam 724C, as desired, to generate a third guide beam 724B3 that is directed toward the third energy guide 722A3, a fourth guide beam 724B4 that is directed toward the fourth energy guide 722A4, and a fifth guide beam 724B5 that is directed toward the fifth energy guide 722A5.

Each AOD 728A, 728B can be designed in a similar manner to those described in greater detail above. For example, the first AOD 728A can include a first transducer 772A and a first absorber 774A that cooperate to generate the desired driving frequency that can, in turn, generate the desired deflection angle so that the source beam 724A is redirected as desired; and the second AOD 728B can include a second transducer 772B and a second absorber 774B that cooperate to generate the desired driving frequency that can, in turn, generate the desired deflection angle so that the transmitted beam 724C is redirected as desired. Alternatively, the first AOD 728A and/or the second AOD 728B can have another suitable design.

As with the previous embodiments, it is appreciated that the system controller 726 controlling the multiplexer 728 to achieve the desired firing sequence plays an important role in the generation of the desired advancing wavefront 835 (illustrated in FIG. 8A).

As noted above, the catheter system incorporating features of the present invention can include any suitable number of energy guides, certain examples of which are illustrated and described in relation to FIGS. 8A-13. It is appreciated, however, that the catheter system need not utilize all of the available energy guides included within the catheter system for purposes of generating any desired advancing wavefront 835.

Moreover, while a number of specific examples of the generation of an advancing wavefront 835 are described herein below, it is appreciated that any desired advancing wavefront 835 can be generated through use of the present invention by (1) controlling the multiplexer with the system controller to selectively power any two or more of the energy guides in any desired firing sequence (or otherwise controlling the desired firing sequence without the use of the multiplexer); (2) positioning the guide distal end of each of the energy guides that are to be selectively powered in the desired firing sequence at a desired longitudinal position relative to the length of the balloon; (3) controlling the energy source with the system controller to generate a series of energy pulses having any desired pulse frequency (and which during a given operation can be increasing, decreasing, and/or substantially constant); (4) controlling the energy source with the system controller to generate a series of energy pulses having any desired pulse energy level (and which during a given operation can be increasing, decreasing, and/or substantially constant); and/or (5) controlling the energy source with the system controller to generate a series of energy pulses having any desired pulse width, or pulse duration (and which during a given operation can be increasing, decreasing, and/or substantially constant). Thus, the inclusion herein of only a limited number of specific examples of generation of a suitable and/or desired advancing wavefront 835 is not intended to limit the vast number of possible alternative advancing wavefronts that could be generated using the teaching of the present invention.

As described herein, in various embodiments and implementations of the present invention, it is appreciated that the direction and focus of the advancing wavefront 835 that is being generated is generally controlled by (1) controlling the multiplexer with the system controller to selectively power any two or more of the energy guides in any desired firing sequence (or otherwise controlling the desired firing sequence without the use of the multiplexer); and (2) positioning the guide distal end of each of the energy guides that are to be selectively powered in the desired firing sequence at a desired longitudinal position relative to the length of the balloon.

It is further appreciated that the size, shape and overall energy level of the specific advancing wavefront 835 that is being generated in any particular use of the catheter system is controlled by one or more (or all) of (1) controlling the energy source with the system controller to generate a series of energy pulses having any desired pulse frequency (and which during a given operation can be increasing, decreasing, and/or substantially constant); (2) controlling the energy source with the system controller to generate a series of energy pulses having any desired pulse energy level (and which during a given operation can be increasing, decreasing, and/or substantially constant); and (3) controlling the energy source with the system controller to generate a series of energy pulses having any desired pulse width, or pulse duration (and which during a given operation can be increasing, decreasing, and/or substantially constant).

FIG. 8A is a simplified schematic side view of a portion of another embodiment of the catheter system 800. More specifically, as shown in FIG. 8A, the catheter system 800 includes at least a balloon 804, a catheter shaft 810, a guidewire lumen 818, and a plurality of energy guides 822 which are spaced apart from one another about the circumference of the guidewire lumen 818. The balloon 804, the catheter shaft 810, the guidewire lumen 818 and the plurality of energy guides 822 are generally similar in design and operation to what has been described in detail herein above. Thus, the balloon 804, the catheter shaft 810, the guidewire lumen 818 and the plurality of energy guides 822 will not be described in detail again in relation to the embodiment shown in FIG. 8A.

As with embodiments described in detail above, the catheter system 800, including the energy source 124 (illustrated in FIG. 1) and/or the multiplexer 128 (illustrated in FIG. 1), can be configured and controlled, such as by the system controller 126 (illustrated in FIG. 1), to selectively and/or separately power each of the plurality of energy guides 822 in any desired firing sequence, pattern, order, firing rate and/or firing duration in order to generate a desired advancing wavefront 835 for purposes of imparting pressure onto and inducing fractures in vascular lesions 106A (illustrated in FIG. 1) at a treatment site 106 (illustrated in FIG. 1). As noted above, FIG. 8A illustrates an exemplary, representative advancing wavefront 835 that could be generated in the manner described using the system controller 126 to control various aspects of the energy source 124 and/or the multiplexer 128. As illustrated, the advancing wavefront 835 is moving generally along the length 842 of the balloon 804 from near the balloon proximal end 804P toward the balloon distal end 804D.

It is appreciated that although each of the plurality of energy guides 822 can be powered separately in any desired firing sequence, pattern, order, firing rate and/or firing duration, sets and/or subsets of the plurality of energy guides 822 can also be powered at any given point in time. Each set or subset of the plurality of energy guides 822 can include one or more of the plurality of energy guides 822. Thus, at any given point in time, power can be directed to one or more of the plurality of energy guides 822 to alternatively create a first firing sequence, a second firing sequence, a third firing sequence, a fourth firing sequence, etc. Moreover, although not required, one or more of the firing sequences of the energy guides 822 in such sets and subsets of the plurality of energy guides 822 can be different than any or all of the other firing sequences of the energy guides 822.

Additionally, during the use of the catheter system 800 to generate a desired advancing wavefront 835, the system controller 126 can further be utilized to control the energy source 124 to generate energy pulses having any desired pulse frequency, pulse energy level, and/or pulse width (or pulse duration). Such control of the energy source 124 with the system controller 126 can help control the specific type (including size, shape and overall energy level) of advancing wavefront 835 that is being generated. More specifically, as noted above, different types of advancing wavefronts can be generated by using a combination of (i) increasing pulse frequency and increasing pulse energy level; (ii) increasing pulse frequency and decreasing pulse energy level; (iii) increasing pulse frequency and constant pulse energy level; (iv) decreasing pulse frequency and increasing pulse energy level; (v) decreasing pulse frequency and decreasing pulse energy level; (vi) decreasing pulse frequency and constant pulse energy level; (vii) constant pulse frequency and increasing pulse energy level; (viii) constant pulse frequency and decreasing pulse energy level; and/or (ix) constant pulse frequency and constant pulse energy level. The types of advancing wavefronts can further be modified from the noted combinations by using pulse widths that are either increasing, decreasing and/or constant with any of such combinations.

FIG. 8B is a simplified schematic cross-sectional view of the portion of the catheter system 800 taken on line B-B in FIG. 8A. More particularly, FIG. 8B again illustrates the balloon 804, the catheter shaft 810, the guidewire lumen 818, and the plurality of energy guides 822 that can be included within this embodiment of the catheter system 800. Further, as shown, FIG. 8B illustrates that this particular non-exclusive embodiment of the catheter system 800 includes eight energy guides, including a first energy guide 822A1, a second energy guide 822A2, a third energy guide 822A3, a fourth energy guide 822A4, a fifth energy guide 822A5, a sixth energy guide 822A6, a seventh energy guide 822A7, and an eighth energy guide 822A8. It is understood, however, that any suitable number of energy guides can be used for any given treatment procedure. More specifically, although the catheter system 800 as shown in FIG. 8B includes eight energy guides 822A1-822A8, a suitable and/or desired advancing wavefront 835 can be generated by using or firing any two, three, four, five, six, seven or eight of the available energy guides 822A1-822A8. It is further appreciated that the catheter system 800 can include a different number of energy guides than what is shown in FIG. 8B, either fewer than eight energy guides or greater than eight energy guides.

In FIG. 8B, the energy guides 822A1-822A8 are uniformly separated by about 45 degrees from one another around the circumference of the guidewire lumen 818. However, it is appreciated that the energy guides 822A1-822A8 need not be uniformly separated from one another, such that the energy guides 822A1-822A8 can be non-uniformly separated from one another, around the circumference of the guidewire lumen 818.

Referring again to FIG. 8A, each of the energy guides 822A1-822A8 includes a guide distal end 822D that can be positioned in any suitable or desired longitudinal position relative to a length 842 of the balloon 804 and/or a length of the guidewire lumen 818 to more effectively and precisely generate the desired advancing wavefront 835 for purposes of imparting pressure waves in order to disrupt the vascular lesions 106A at the treatment site 106. For example, the first energy guide 822A1 can include a first guide distal end 822D that is positioned at a first longitudinal position relative to the length 842 of the balloon 804; the second energy guide 822A2 can include a second guide distal end 822D that is positioned at a second longitudinal position relative to the length 842 of the balloon 804; the third energy guide 822A3 can include a third guide distal end 822D that is positioned at a third longitudinal position relative to the length 842 of the balloon 804; the fourth energy guide 822A4 can include a fourth guide distal end 822D that is positioned at a fourth longitudinal position relative to the length 842 of the balloon 804; the fifth energy guide 822A5 can include a fifth guide distal end 822D that is positioned at a fifth longitudinal position relative to the length 842 of the balloon 804; the sixth energy guide 822A6 can include a sixth guide distal end 822D that is positioned at a sixth longitudinal position relative to the length 842 of the balloon 804; the seventh energy guide 822A7 can include a seventh guide distal end 822D that is positioned at a seventh longitudinal position relative to the length 842 of the balloon 804; and the eighth energy guide 822A8 can include an eighth guide distal end 822D that is positioned at an eighth longitudinal position relative to the length 842 of the balloon 804.

It is appreciated that, in alternative embodiments, each of the longitudinal positions of the guide distal ends 822D relative to the length 842 of the balloon 804 can be different than one another, or two or more of the longitudinal positions of the guide distal ends 822D relative to the length 842 of the balloon 804 can be the same as one another. Although each of the energy guides 822A1-822A8 is shown as being positioned substantially directly adjacent to the guidewire lumen 818, it is recognized that a portion of the energy guide 822A1-822A8, such as the guide distal end 822D, can be spaced apart from the guidewire lumen 818. For example, the guide distal end 822D of any of the energy guides 822A1-822A8 can be located at any suitable position laterally between the guidewire lumen 818 and the balloon wall 830 of the balloon 804.

In various embodiments and implementations, as described herein, it is possible to fire individual energy guides 822A1-822A8, and/or sets or subsets of the energy guides 822A1-822A8, to achieve a desired firing sequence or pattern, in combination with controlling the pulse frequency, the pulse energy level and/or the pulse width of the energy pulses from the energy source 124, that could be effective for generating a desired advancing wavefront 835 for purposes of disrupting localized calcified lesions. More specifically, the system controller 126 can control the energy source 124 and/or the multiplexer 128 so that individual guide beams 124B (illustrated in FIG. 1) can be directed to any of the energy guides 822A1-822A8, or sets or subsets of the energy guides 822A1-822A8, in any desired firing sequence, pattern, order, firing rate and/or firing duration, and with any desired pulse frequency, pulse energy level and/or pulse width, to achieve a greater degree of disruption of the calcified lesions. For example, with eight energy guides 822A1-822A8 that are arranged in a linear pattern with angular orientation spiraling around the guidewire lumen 818, the system controller 126 can control the firing sequence of the light energy from the energy source 124 to any two or more of the energy guides 822A1-822A8 in any desired predetermined or non-predetermined manner.

A non-exclusive listing of exemplary implementations for generating suitable or desired advancing wavefronts utilizing the present invention will now be described. It is appreciated, however, that one skilled in the relevant art would easily identify alternative implementations that could further be utilized to generate other suitable or desired advancing wavefronts based on the specific teaching provided herein.

In one non-exclusive implementation, the system controller 126 can direct individual guide beams 124B to each of the first energy guide 822A1 and the eighth energy guide 822A8 in a first set of energy guides, then having individual guide beams 124B directed to each of the second energy guide 822A2 and the seventh energy guide 822A7 in a second set of energy guides, followed by individual guide beams 124B directed to each of the third energy guide 822A3 and the sixth energy guide 822A6 in a third set of energy guides, and finally having individual guide beams 124B directed to each of the fourth energy guide 822A4 and the fifth energy guide 822A5 in a fourth set of energy guides. This example of a firing sequence and/or firing pattern generates a pair of advancing wavefronts 835, with one advancing wavefront 835 converging from each end of the balloon 804 toward a specific region such as the treatment site 106, located between the guide distal ends 822D of the fourth energy guide 822A4 and the fifth energy guide 822A5, and can thereby more effectively disrupt a lesion at that location. As described, each of the noted advancing wavefronts 835 has been generated using four of the available eight energy guides 822A1-822A8. Each of the generated advancing wavefronts 835 can be further tailored and/or modified as desired by using the system controller 126 to control the energy source 124 to generate energy pulses having a given pulse frequency, pulse energy level, and/or pulse width. Moreover, each of the pulse frequency, the pulse energy level, and the pulse width can be increasing, decreasing or substantially constant as each successive energy guide 822A1-822A8 is fired in the noted firing sequences.

In another non-exclusive implementation, the system controller 126 can control the energy source 124 and/or the multiplexer 128 to direct individual guide beams 124B to each of the third energy guide 822A3 and the seventh energy guide 822A7 in a first set of energy guides, followed by individual guide beams 124B directed to each of the fourth energy guide 822A4 and the sixth energy guide 822A6 in a second set of energy guides, and finally having an individual guide beam 124B directed to the fifth energy guide 822A5 in a third set of energy guides. This example of a firing sequence and/or firing pattern again generates a pair of advancing wavefronts 835, with one advancing wavefront 835 converging from each end of the balloon 804 toward a specific region (such as the treatment site 106, in one embodiment) located at a longitudinal position, such as the fifth longitudinal position, within the balloon 804 near the guide distal end 822D of the fifth energy guide 822A5. As described, each of the noted advancing wavefronts 835 has been generated using three of the available eight energy guides 822A1-822A8. As with the previously noted example, and with all other examples described herein, each of the generated advancing wavefronts 835 can be further tailored and/or modified as desired by using the system controller 126 to control the energy source 124 to generate energy pulses having a given pulse frequency, pulse energy level, and/or pulse width, which can each be increasing, decreasing or substantially constant as each successive energy guide 822A1-822A8 is fired in the noted firing sequences.

In still another non-exclusive implementation, the system controller 126 can control the energy source 124 and/or the multiplexer 128 to generate somewhat different advancing wavefronts 835 along substantially a full length 842 of the balloon 804. In this embodiment, the system controller 126 can control the energy source 124 and/or the multiplexer 128 to sequentially direct individual guide beams 124B to the first energy guide 822A1, the second energy guide 822A2, and the third energy guide 822A3 in a first sequence to generate a first advancing wavefront 835. Next, the system controller 126 can control the energy source 124 and/or the multiplexer 128 to sequentially direct individual guide beams 124B to the second energy guide 822A2, the third energy guide 822A3, and the fourth energy guide 822A4 in a second sequence to generate a second advancing wavefront 835. This type of sequencing can continue in a similar manner, (such as third, fourth, then fifth in a third sequence to generate a third advancing wavefront 835; fourth, fifth, then sixth in a fourth sequence to generate a fourth advancing wavefront 835; fifth, sixth, then seventh in a fifth sequence to generate a fifth advancing wavefront 835, etc.), until a last step when the system controller 126 controls the energy source 124 and/or the multiplexer 128 to sequentially direct individual guide beams 124B to the sixth energy guide 822A6, the seventh energy guide 822A7, and the eighth energy guide 822A8 in a sixth sequence to generate a sixth advancing wavefront 835. It is further appreciated that each of the advancing wavefronts 835 can be combined to form a composite advancing wavefront.

In another non-exclusive implementation, the system controller 126 can control the energy source 124 and/or the multiplexer 128 to direct individual guide beams 124B to each of the energy guides 822A1-822A8 substantially along the full length 842 of the balloon 804. More specifically, the balloon 804 can be positioned such that the eighth energy guide 822A8 is positioned closest to the vascular lesions 106A at the treatment site 106, and the system controller 126 can control the energy source 124 and/or the multiplexer 128 to direct a first guide beam 124B to the first energy guide 822A1, then a second guide beam 124B to the second energy guide 822A2, then a third guide beam 124B to the third energy guide 822A3, then a fourth guide beam 124B to the fourth energy guide 822A4, then a fifth guide beam 124B to the fifth energy guide 822A5, then a sixth guide beam 124B to the sixth energy guide 822A6, then a seventh guide beam 124B to the seventh energy guide 822A7, and then finally an eighth guide beam 124B to the eighth energy guide 822A8, in order to generate an advancing wavefront 835 that moves more fully along the length 842 of the balloon 804.

In yet other non-exclusive implementations, the system controller 126 can control the energy source 124 and/or the multiplexer 128 to direct individual guide beams 124B successively and/or sequentially to any three of the energy guides 822A1-822A8. For example, the system controller 126 can control the energy source 124 and/or the multiplexer 128 to direct individual guide beams 124B successively to (A) the first energy guide 822A1, then to the second energy guide 822A2, and then to the third energy guide 822A3; (B) to the second energy guide 822A2, then to the fourth energy guide 822A4, and then to the fifth energy guide 822A5; and/or (C) to the first energy guide 822A1, then to the fourth energy guide 822A4, and then to the seventh energy guide 822A7. It is appreciated that the particular three energy guides 822A1-822A8 utilized in any such firing sequence can be any of the energy guides 822A1-822A8 so long as they successively have guide distal ends 822D that are closer to the vascular lesions 106A at the treatment site 106. Moreover, it is further appreciated that the generated advancing wavefront835 can move in either direction toward the vascular lesions 106A at the treatment site 106, i.e. either left-to-right or right-to-left as shown in FIG. 8A, such that the energy guides 822A1-822A8 are used in an increasing number order (such as first-second-third) or a decreasing number order (such as eighth-seventh-sixth).

In still other non-exclusive implementations, the system controller 126 can control the energy source 124 and/or the multiplexer 128 to direct individual guide beams 124B successively and/or sequentially to any four of the energy guides 822A1-822A8, to any five of the energy guides 822A1-822A8, to any six of the energy guides 822A1-822A8, or to any seven of the energy guides 822A1-822A8.

In other non-exclusive implementations, the system controller 126 can control the energy source 124 and/or the multiplexer 128 to direct individual guide beams 124B successively and/or sequentially to any two of the energy guides 822A1-822A8.

As noted above, in certain implementations, the guide distal end 822D of more than one energy guide 822A1-822A8 can be located at any given longitudinal position relative to the length 842 of the balloon 804. For example, in some such implementations, two of the eight energy guides 822A1-822A8 can be located at each of four different longitudinal positions relative to the length 842 of the balloon 804. With such arrangement, in one implementation, the system controller 126 can control the energy source 124 such that both energy guides having their guide distal end at a first longitudinal position can be fired first, then both energy guides having their guide distal end at a second longitudinal position can be fired second, then both energy guides having their guide distal end at a third longitudinal position can be fired third, and then both energy guides having their guide distal end at a fourth longitudinal position can be fired last. This can still function to generate a desired advancing wavefront 835, but one which can have greater circumferential coverage about the balloon 804.

It is further noted that the guide distal end 822D of each energy guide 822A1-822A8 can be positioned at any suitable location about the guidewire lumen 818. Depending on such positioning of the guide distal end 822D of each of the energy guides 822A1-822A8, and the specific firing sequence being implemented, this can impact the overall direction of the advancing wavefront 835. For example, if the guide distal end 822D of successively fired energy guides 822A1-822A8 are positioned in different locations gradually about the guidewire lumen 818, then the advancing wavefront 835 can further tend to exhibit a spiral-type shape. However, other desired modifications of the shape and/or orientation of the advancing wavefront 835 can also be realized depending on the specific positioning of the guide distal end 822D of successively fired energy guides 822A1-822A8 about the guidewire lumen 818 and/or relative to the length 842 of the balloon 804.

It is appreciated that in all of the specific examples and implementations noted above, the specific type, size, shape and overall energy level of the generated advancing wavefront 835 can be tailored and/or modified as desired by utilizing the system controller 126 to control the energy source 124 and/or the multiplexer 128 to control (i) the number of energy guides being utilized, (ii) the firing sequence for the energy guides being utilized, (iii) the longitudinal positioning of the guide distal end of the energy guides being utilized, (iv) the pulse frequency of the pulses of energy directed to the energy guides (which can be increasing, decreasing, and/or constant), (v) the pulse energy level of the pulses of energy directed to the energy guides (which can be increasing, decreasing, and/or constant), and/or (vi) the pulse width of the pulses of energy directed to the energy guides (which can be increasing, decreasing, and/or constant). Additionally, or in the alternative, the advancing wavefront 835 can be further tailored and/or modified by controlling other suitable energy parameters.

In various embodiments, the advancing wavefront 835 need not necessarily solely advance toward the treatment site 106, but may represent a wavefront that advances toward the treatment site 106 and subsequently continues past the treatment site 106 (in any suitable direction).

In many embodiments, the advancing wavefront 835 is spherical-shaped and impinges at an angle relative to the balloon wall 830 to create a shearing force at the balloon wall 830 substantially adjacent to the treatment site 106. More particularly, in some embodiments, the system controller 126 can control the energy source 124 and/or the multiplexer 128 to create an advancing series of energy waves along the length 842 of the balloon 804 for creating a shear wave in the vascular lesions 106A at the treatment site 106. In such applications, as individual guide beams 124B are directed to specific energy guides 822A1-822A8 in a specific sequence, the pressure waves will advance in the direction of activation. As new bubbles are created ahead of collapsing ones, it would be possible to create a shearing force at the balloon wall 830. The localized force on the leading edge of the spherical wavefront impinging at an angle relative to the balloon wall 830 that is non-normal creates a highly concentrated, localized shearing force. This could have a greater effect in cracking calcified lesions compared to simply hitting the walls through the length 842 of the balloon 804 with one radially directed pressure wave, which can expand the whole cross-section of the balloon 804 creating hoop stress.

In certain embodiments, it may be desirable to have the guide distal end 822D of more than one energy guide 822A1-822A8 be positioned at the same longitudinal position relative to the length 842 of the balloon 804, and the system controller 126 can control the energy source 124 and/or the multiplexer 128 to fire each of such energy guides 822A1-822A8 substantially simultaneously to generate pressure waves fully about the balloon 804 at such longitudinal position.

It is also appreciated that the foregoing examples of embodiments describing and/or illustrating particular firing sequences or patterns are provided as representative examples only, and are not intended to be limiting in any manner. In fact, it is further appreciated that an unlimited number of different such firing sequences and/or patterns can be achieved utilizing the disclosure provided herein. It is appreciated that with any of the embodiments shown, described and/or achievable using the disclosure herein, that the firing rate can be controlled so that the firing rate increases or decreases over time. The firing rate can be controlled so that the firing rate increases or decreases depending upon the specific energy guides to which light energy is being directed.

It is further recognized that with the designs provided herein, any desired firing sequence and/or pattern can be achieved. The types of firing sequences and/or patterns that can be achieved can be based at least in part on the number of energy guides, the axial and longitudinal positioning of each of the energy guides within the balloon, the energy level of the firing of each of the energy guides, the rate of firing, etc. It is understood that by controlling these and any other suitable parameters, an advancing wavefront 835 resulting in a gradual or abrupt disruption of the calcification of a vascular lesion can occur. With these designs, the likelihood of success for adequate and/or satisfactory disruption of the calcification in a vascular lesion and/or heart valve is increased.

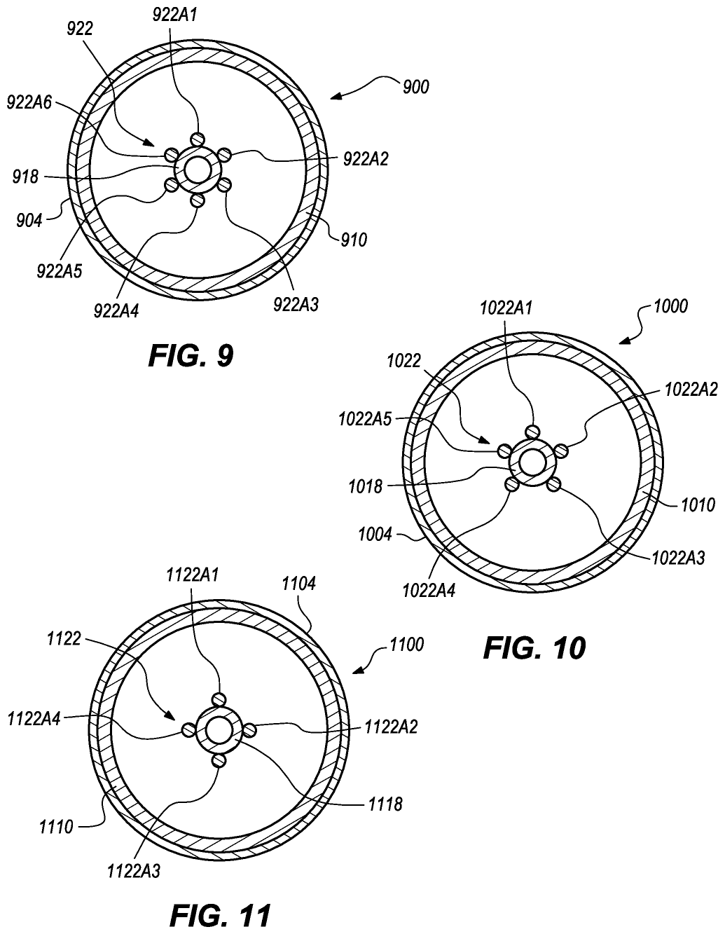

FIG. 9 is a simplified schematic cross-sectional view of another embodiment of the catheter system 900. The catheter system 900 illustrated in FIG. 9 is similar to the catheter system 800 illustrated in FIGS. 8A and 8B, except that the catheter system 900 in FIG. 9 includes a different number of energy guides. More particularly, FIG. 9 illustrates that the catheter system 900 includes at least a balloon 904, a catheter shaft 910, a guidewire lumen 918, and a plurality of energy guides 922, such as a first energy guide 922A1, a second energy guide 922A2, a third energy guide 922A3, a fourth energy guide 922A4, a fifth energy guide 922A5, and a sixth energy guide 922A6, which are uniformly separated by about 60 degrees from one another around the circumference of the guidewire lumen 918. As with previous embodiments, it is appreciated that the energy guides 922A1-922A6 need not be uniformly separated from one another around the circumference of the guidewire lumen 918.

As with the previous embodiments, each of the energy guides 922A1-922A6 can include a guide distal end (not shown in FIG. 9) that can be positioned at any desired longitudinal position relative to a length of the balloon 904 and/or relative to a length of the guidewire lumen 918. It is also appreciated that the system controller 126 (illustrated in FIG. 1) can control the energy source 124 (illustrated in FIG. 1) and/or the multiplexer 128 (illustrated in FIG. 1) so that light energy is separately directed to any two or more of the energy guides 922A1-922A6 in any desired firing sequence or pattern, such as to generate any desired advancing wavefront 835 (illustrated in FIG. 8A). Also similar to previous embodiments, the system controller 126 can control the energy source 124 to generate energy pulses having any desired pulse frequency (increasing, decreasing or constant), pulse energy level (increasing, decreasing or constant), and pulse width (increasing, decreasing or constant).

FIG. 10 is a simplified schematic cross-sectional view of still another embodiment of the catheter system 1000. The catheter system 1000 illustrated in FIG. 10 is similar to the catheter systems illustrated and described herein above, except that the catheter system 1000 in FIG. 10 includes a different number of energy guides. More particularly, FIG. 10 illustrates that the catheter system 1000 includes at least a balloon 1004, a catheter shaft 1010, a guidewire lumen 1018, and a plurality of energy guides 1022, such as a first energy guide 1022A1, a second energy guide 1022A2, a third energy guide 1022A3, a fourth energy guide 1022A4, and a fifth energy guide 1022A5, which are uniformly separated by about 72 degrees from one another around the circumference of the guidewire lumen 1018. As with previous embodiments, it is appreciated that the energy guides 1022A1-1022A5 need not be uniformly separated from one another around the circumference of the guidewire lumen 1018.

As with the previous embodiments, each of the energy guides 1022A1-1022A5 can include a guide distal end (not shown in FIG. 10) that can be positioned at any desired longitudinal position relative to a length of the balloon 1004 and/or relative to a length of the guidewire lumen 1018. It is also appreciated that the system controller 126 (illustrated in FIG. 1) can control the energy source 124 (illustrated in FIG. 1) and/or the multiplexer 128 (illustrated in FIG. 1) so that light energy is separately directed to any two or more of the energy guides 1022A1-1022A5 in any desired firing sequence or pattern, such as to generate any desired advancing wavefront 835 (illustrated in FIG. 8A). Also similar to previous embodiments, the system controller 126 can control the energy source 124 to generate energy pulses having any desired pulse frequency (increasing, decreasing or constant), pulse energy level (increasing, decreasing or constant), and pulse width (increasing, decreasing or constant).

FIG. 11 is a simplified schematic cross-sectional view of yet another embodiment of the catheter system 1100. The catheter system 1100 illustrated in FIG. 11 is similar to the catheter systems illustrated and described herein above, except that the catheter system 1100 in FIG. 11 includes a different number of energy guides. More particularly, FIG. 11 illustrates that the catheter system 1100 includes at least a balloon 1104, a catheter shaft 1110, a guidewire lumen 1118, and a plurality of energy guides 1122, such as a first energy guide 1122A1, a second energy guide 1122A2, a third energy guide 1122A3, and a fourth energy guide 1122A4, which are uniformly separated by about 90 degrees from one another around the circumference of the guidewire lumen 1118. As with previous embodiments, it is appreciated that the energy guides 1122A1-1122A4 need not be uniformly separated from one another around the circumference of the guidewire lumen 1118.

As with the previous embodiments, each of the energy guides 1122A1-1122A4 can include a guide distal end (not shown in FIG. 11) that can be positioned at any desired longitudinal position relative to a length of the balloon 1104 and/or relative to a length of the guidewire lumen 1118. It is also appreciated that the system controller 126 (illustrated in FIG. 1) can control the energy source 124 (illustrated in FIG. 1) and/or the multiplexer 128 (illustrated in FIG. 1) so that light energy is separately directed to any two or more of the energy guides 1122A1-1122A4 in any desired firing sequence or pattern, such as to generate any desired advancing wavefront 835 (illustrated in FIG. 8A). Also similar to previous embodiments, the system controller 126 can control the energy source 124 to generate energy pulses having any desired pulse frequency (increasing, decreasing or constant), pulse energy level (increasing, decreasing or constant), and pulse width (increasing, decreasing or constant).

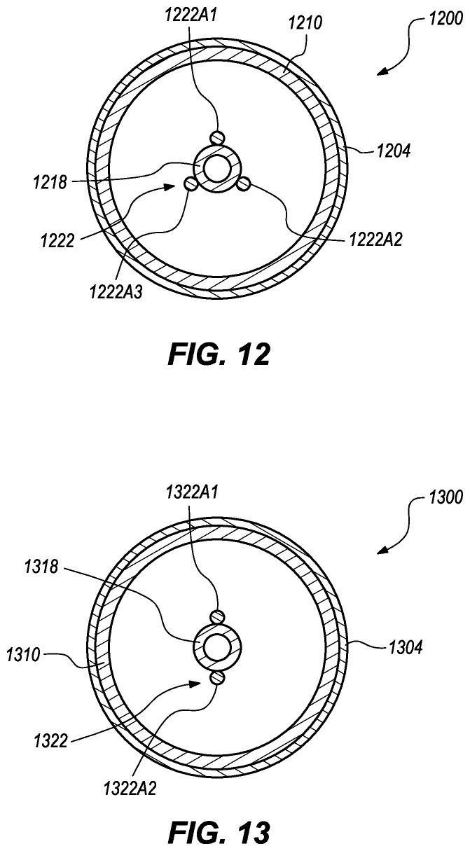

FIG. 12 is a simplified schematic cross-sectional view of still another embodiment of the catheter system 1200. The catheter system 1200 illustrated in FIG. 12 is similar to the catheter systems illustrated and described herein above, except that the catheter system 1200 in FIG. 12 includes a different number of energy guides. More particularly, FIG. 12 illustrates that the catheter system 1200 includes at least a balloon 1204, a catheter shaft 1210, a guidewire lumen 1218, and a plurality of energy guides 1222, such as a first energy guide 1222A1, a second energy guide 1222A2, and a third energy guide 1222A3, which are uniformly separated by about 120 degrees from one another around the circumference of the guidewire lumen 1218. As with previous embodiments, it is appreciated that the energy guides 1222A1-1222A3 need not be uniformly separated from one another around the circumference of the guidewire lumen 1218.

As with the previous embodiments, each of the energy guides 1222A1-1222A3 can include a guide distal end (not shown in FIG. 12) that can be positioned at any desired longitudinal position relative to a length of the balloon 1204 and/or relative to a length of the guidewire lumen 1218. It is also appreciated that the system controller 126 (illustrated in FIG. 1) can control the energy source 124 (illustrated in FIG. 1) and/or the multiplexer 128 (illustrated in FIG. 1) so that light energy is separately directed to any two or more of the energy guides 1222A1-1222A3 in any desired firing sequence or pattern, such as to generate any desired advancing wavefront 835 (illustrated in FIG. 8A). Also similar to previous embodiments, the system controller 126 can control the energy source 124 to generate energy pulses having any desired pulse frequency (increasing, decreasing or constant), pulse energy level (increasing, decreasing or constant), and pulse width (increasing, decreasing or constant).

FIG. 13 is a simplified schematic cross-sectional view of still yet another embodiment of the catheter system 1300. The catheter system 1300 illustrated in FIG. 13 is similar to the catheter systems illustrated and described herein above, except that the catheter system 1300 in FIG. 13 includes a different number of energy guides. More particularly, FIG. 13 illustrates that the catheter system 1300 includes at least a balloon 1304, a catheter shaft 1310, a guidewire lumen 1318, and a plurality of energy guides 1322, such as a first energy guide 1322A1, and a second energy guide 1322A2, which are uniformly separated by about 180 degrees from one another around the circumference of the guidewire lumen 1318. As with previous embodiments, it is appreciated that the energy guides 1322A1-1322A2 need not be uniformly separated from one another around the circumference of the guidewire lumen 1318.

As with the previous embodiments, each of the energy guides 1322A1-1322A2 can include a guide distal end (not shown in FIG. 13) that can be positioned at any desired longitudinal position relative to a length of the balloon 1304 and/or relative to a length of the guidewire lumen 1318. It is also appreciated that the system controller 126 (illustrated in FIG. 1) can control the energy source 124 (illustrated in FIG. 1) and/or the multiplexer 128 (illustrated in FIG. 1) so that light energy is separately directed to each of the energy guides 1322A1-1322A2 in any desired firing sequence or pattern, such as to generate any desired advancing wavefront 835 (illustrated in FIG. 8A). Also similar to previous embodiments, the system controller 126 can control the energy source 124 to generate energy pulses having any desired pulse frequency (increasing, decreasing or constant), pulse energy level (increasing, decreasing or constant), and pulse width (increasing, decreasing or constant).

FIGS. 14A-14H are graphical representations of combinations of pulse frequency and pulse energy level that can be controlled by the system controller for purposes of generating desired alternative types of advancing wavefronts.

In particular, FIG. 14A is a graphical representation which illustrates a combination of an increasing pulse frequency and an increasing pulse energy level that can be implemented for successive pulses in a desired firing sequence to generate a first type of advancing wavefront. As shown, the pulse frequency and the pulse energy level are increasing at a fairly constant rate relative to one another. Alternatively, in other implementations, the pulse frequency can increase at a faster rate than the pulse energy level, or the pulse energy level can increase at a faster rate than the pulse frequency.

FIG. 14B is a graphical representation which illustrates a combination of an increasing pulse frequency and a decreasing pulse energy level that can be implemented for successive pulses in a desired firing sequence to generate a second type of advancing wavefront. As shown, the pulse frequency is increasing and the pulse energy level is decreasing at fairly constant rates relative to one another. Alternatively, in other implementations, the pulse frequency can increase at a faster rate than the pulse energy level is decreasing, or the pulse energy level can decrease at a faster rate than the pulse frequency is increasing.

FIG. 14C is a graphical representation which illustrates a combination of an increasing pulse frequency and a substantially constant pulse energy level that can be implemented for successive pulses in a desired firing sequence to generate a third type of advancing wavefront.

FIG. 14D is a graphical representation which illustrates a combination of a decreasing pulse frequency and an increasing pulse energy level that can be implemented for successive pulses in a desired firing sequence to generate a fourth type of advancing wavefront. As shown, the pulse frequency is decreasing and the pulse energy level is increasing at fairly constant rates relative to one another. Alternatively, in other implementations, the pulse frequency can decrease at a faster rate than the pulse energy level is increasing, or the pulse energy level can increase at a faster rate than the pulse frequency is decreasing.

FIG. 14E is a graphical representation which illustrates a combination of a decreasing pulse frequency and a decreasing pulse energy level that can be implemented for successive pulses in a desired firing sequence to generate a fifth type of advancing wavefront. As shown, the pulse frequency and the pulse energy level are decreasing at a fairly constant rate relative to one another. Alternatively, in other implementations, the pulse frequency can decrease at a faster rate than the pulse energy level, or the pulse energy level can decrease at a faster rate than the pulse frequency.

FIG. 14F is a graphical representation which illustrates a combination of a decreasing pulse frequency and a substantially constant pulse energy level that can be implemented for successive pulses in a desired firing sequence to generate a sixth type of advancing wavefront.

FIG. 14G is a graphical representation which illustrates a combination of a substantially constant pulse frequency and an increasing pulse energy level that can be implemented for successive pulses in a desired firing sequence to generate a seventh type of advancing wavefront.

FIG. 14H is a graphical representation which illustrates a combination of a substantially constant pulse frequency and a decreasing pulse energy level that can be implemented for successive pulses in a desired firing sequence to generate an eighth type of advancing wavefront.

As described in detail herein, in various embodiments, the present invention can be utilized to solve various problems that exist in more traditional catheter systems. For example, by enabling the catheter system to fire each energy guide separately, it is possible to achieve a sequence or firing sequence that could be much more effective at breaking localized lesions. In many embodiments, firing individual energy guides in a desired firing sequence or pattern can create a moving or advancing energy wavefront that more effectively breaks up a lesion at one particular location or an extended lesion. In certain embodiments, the separate firing of the individual energy guides can be utilized to create a localized shearing force on the leading edge of the spherical wavefront that impinges at an angle (non-normal) relative to the balloon wall, which could have a greater effect in cracking calcified lesions.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the detailed description provided herein. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope, and no limitations are intended to the details of construction or design herein shown.

What is claimed is:

1. A catheter system for treating a treatment site within or adjacent to a vessel wall of a blood vessel, the catheter system comprising:
a catheter shaft;
a balloon that is coupled to the catheter shaft, the balloon includes a balloon first end, a balloon second end, and a balloon wall that defines a balloon interior, the balloon wall being configured to be positioned adjacent to the treatment site;
an energy source that generates a plurality of pulses of energy;
a plurality of energy guides that are each configured to selectively receive at least one of the plurality of pulses of energy from the energy source, each of the plurality of energy guides being disposed along the catheter shaft and at least partially within the balloon interior, the plurality of energy guides each including a corresponding guide distal end, each of the guide distal ends being positioned within the balloon interior at a different longitudinal position from one another along a length of the balloon; and
a system controller that (i) controls at least one of a pulse frequency, a pulse energy level and a pulse width of each of the plurality of pulses of energy from the energy source, and (ii) controls a firing sequence of the plurality of pulses of energy from the energy source such that the plurality of pulses of energy from the energy source are sequentially directed to each of the plurality of energy guides, so that an advancing wavefront is generated within the balloon interior that moves toward the treatment site in a first direction from near the balloon first end toward the balloon second end.

2. The catheter system of claim 1 wherein the system controller controls at least two of the pulse frequency, the pulse energy level and the pulse width of each of the plurality of pulses of energy from the energy source, so that the advancing wavefront is generated within the balloon interior that moves toward the treatment site in the first direction from near the balloon first end toward the balloon second end.

3. The catheter system of claim 2 wherein the system controller controls each of the pulse frequency, the pulse energy level and the pulse width of each of the plurality of pulses of energy from the energy source, so that the advancing wavefront is generated within the balloon interior that moves toward the treatment site in the first direction from near the balloon first end toward the balloon second end.

4. The catheter system of claim 1 wherein the system controller controls the pulse frequency so that the pulse frequency is increasing during the firing sequence of the plurality of pulses of energy from the energy source.

5. The catheter system of claim 1 wherein the system controller controls the pulse frequency so that the pulse frequency is decreasing during the firing sequence of the plurality of pulses of energy from the energy source.

6. The catheter system of claim 1 wherein the system controller controls the pulse energy level so that the pulse energy level is increasing during the firing sequence of the plurality of pulses of energy from the energy source.

7. The catheter system of claim 1 wherein the system controller controls the pulse energy level so that the pulse energy level is decreasing during the firing sequence of the plurality of pulses of energy from the energy source.

8. The catheter system of claim 1 wherein the system controller controls the energy source so that the pulse frequency is increasing and the pulse energy level of each of the plurality of pulses of energy is increasing during the firing sequence, so that the advancing wavefront is generated within the balloon interior that moves toward the treatment site in the first direction from near the balloon first end toward the balloon second end.

9. The catheter system of claim 1 wherein the system controller controls the energy source so that the pulse frequency is increasing and the pulse energy level of each of the plurality of pulses of energy is decreasing during the firing sequence, so that the advancing wavefront is generated within the balloon interior that moves toward the treatment site in the first direction from near the balloon first end toward the balloon second end.

10. The catheter system of claim 1 wherein the system controller controls the energy source so that the pulse frequency is decreasing and the pulse energy level of each of the plurality of pulses of energy is increasing during the firing sequence, so that the advancing wavefront is generated within the balloon interior that moves toward the treatment site in the first direction from near the balloon first end toward the balloon second end.

11. The catheter system of claim 1 wherein the system controller controls the energy source so that the pulse frequency is decreasing and the pulse energy level of each of the plurality of pulses of energy is decreasing during the firing sequence, so that the advancing wavefront is generated within the balloon interior that moves toward the treatment site in the first direction from near the balloon first end toward the balloon second end.

12. The catheter system of claim 1 further comprising a multiplexer that receives the plurality of pulses of energy from the energy source; and wherein the system controller controls the multiplexer such that the plurality of pulses of energy from the energy source are sequentially directed to each of the plurality of energy guides in accordance with the firing sequence.

13. The catheter system of claim 1 wherein the plurality of energy guides includes a first energy guide, a second energy guide and a third energy guide that are each disposed along the catheter shaft and at least partially within the balloon interior, (i) the first energy guide including a first guide distal end that is positioned within the balloon interior at a first longitudinal position along the length of the balloon, (ii) the second energy guide including a second guide distal end that is positioned within the balloon interior at a second longitudinal position along the length of the balloon that is different than the first longitudinal position, and (iii) the third energy guide including a third guide distal end that is positioned within the balloon interior at a third longitudinal position along the length of the balloon that is different than the first longitudinal position and the second longitudinal position, the second longitudinal position being between the first longitudinal position and the third longitudinal position along the length of the balloon; and wherein the system controller controls the energy source such that that the plurality of pulses of energy from the energy source are sequentially directed to the first energy guide, then the second energy guide, and then the third energy guide so that the advancing wavefront is generated within the balloon interior that moves toward the treatment site in the first direction from near the balloon first end toward the balloon second end.

14. The catheter system of claim 13 wherein the plurality of energy guides further includes a fourth energy guide that is disposed along the catheter shaft and at least partially within the balloon interior, the fourth energy guide including a fourth guide distal end that is positioned within the balloon interior at a fourth longitudinal position along the length of the balloon that is different than the first longitudinal position, the second longitudinal position and the third longitudinal position, the third longitudinal position being between the second longitudinal position and the fourth longitudinal position along the length of the balloon.

15. The catheter system of claim 14 wherein the system controller controls the energy source such that the plurality of pulses of energy from the energy source are sequentially directed to the first energy guide, then the second energy guide, then the third energy guide, and then the fourth energy guide so that the advancing wavefront is generated within the balloon interior that moves toward the treatment site in the first direction from near the balloon first end toward the balloon second end.

16. The catheter system of claim 14 wherein the plurality of energy guides further includes a fifth energy guide that is disposed along the catheter shaft and at least partially within the balloon interior, the fifth energy guide including a fifth guide distal end that is positioned within the balloon interior at a fifth longitudinal position along the length of the balloon that is different than the first longitudinal position, the second longitudinal position, the third longitudinal position and the fourth longitudinal position, the fourth longitudinal position being between the third longitudinal position and the fifth longitudinal position along the length of the balloon.

17. The catheter system of claim 16 wherein the system controller controls the energy source such that the plurality of pulses of energy from the energy source are sequentially directed to the first energy guide, then the second energy guide, then the third energy guide, then the fourth energy guide, and then the fifth energy guide so that the advancing wavefront is generated within the balloon interior that moves toward the treatment site in the first direction from near the balloon first end toward the balloon second end.

18. The catheter system of claim 16 wherein the system controller controls the energy source such that a first plurality of pulses of energy from the energy source are sequentially directed to the first energy guide, then the second energy guide, and then the third energy guide so that the advancing wavefront is generated within the balloon interior that moves toward the treatment site in the first direction from near the balloon first end toward the balloon second end; and wherein the system controller controls the energy source such that a second plurality of pulses of energy from the energy source are sequentially directed to the fifth energy guide, then the fourth energy guide, and then the third energy guide so that a second advancing wavefront is generated within the balloon interior that moves toward the treatment site in a second direction from near the balloon second end toward the balloon first end.

19. The catheter system of claim 1 wherein the advancing wavefront is spherical-shaped and impinges at an angle relative to the balloon wall to create a shearing force at the balloon wall substantially adjacent to the treatment site.

20. The catheter system of claim 1 wherein each of the plurality of energy guides includes an optical fiber; and wherein the energy source is a laser source that generates a plurality of pulses of laser energy.
